(19) United States
(12) Patent Application Publication
(10) Pub. No.: US 2019/0316081 A1
(43) Pub. Date: Oct. 17, 2019

(54) MEDIA AND METHODS FOR EXPANSION OF PLURIPOTENT STEM CELLS

(71) Applicants: Yonatan Y. Lipsitz, Cambridge, MA (US); Peter W. Zandstra, Vancouver (CA)

(72) Inventors: Yonatan Y. Lipsitz, Cambridge, MA (US); Peter W. Zandstra, Vancouver (CA)

(21) Appl. No.: 16/386,247

(22) Filed: Apr. 16, 2019

Related U.S. Application Data
(60) Provisional application No. 62/658,908, filed on Apr. 17, 2018.

(51) Int. Cl. C12N 5/0735; C12N 5/00; C12N 5/0793; C12N 5/077; C12N 5/071

(52) U.S. Cl. CPC C12N 5/0606; C12N 5/0018; C08L 5/02; C12N 5/0657; C12N 5/0676; C12N 5/0619

(57) ABSTRACT

The present disclosure provides methods and reagents for culturing pluripotent stem cells. The method comprises a first step of culturing in a medium comprising inhibitors of GSK-3β, JNK, p38, PKC and Erk1/2, followed by a second step of culturing in a medium comprising inhibitors of GSK-3β, JNK, p38 and PKC, and not comprising an inhibitor of Erk1/2. The medium of the second step may also further comprise dextran sulfate. The pluripotent stem cells produced using the methods and reagents provided may also be differentiated into a cell type of interest. The methods and reagents provided in this disclosure offer robust and scalable technologies for manufacturing the quantities of cells anticipated to be required for widespread patient access.

### CROSS REFERENCE TO PRIOR APPLICATIONS

This application hereby claims the benefit of priority from U.S. Provisional Patent Application No. 62/658,908, filed Apr. 17, 2018, which is incorporated by reference in its entirety.

### FIELD OF THE DISCLOSURE

The present description relates generally to in vitro methods and compositions for culturing pluripotent stem cells. More particularly, the description relates to methods and compositions for inducing the proliferation of pluripotent stem cells.

### BACKGROUND OF THE DISCLOSURE

Pluripotent stem cells (PSC)^{1,2} offer the opportunity to investigate fundamental questions in developmental biology and to advance the development of cell-based therapies (reviewed by Kirouac and Zandstra³). Human (h)PSC can be expanded indefinitely in culture while maintaining the ability to differentiate to all specialized cell types in a manner that parallels some aspects of human development. These qualities render hPSC a particularly promising cell source for developing cell-based therapies to cure a variety of chronic conditions. However, a critical factor that currently limits the successful clinical translation of these therapies is the lack of robust, scalable technologies for manufacturing the quantities of cells anticipated to be required for widespread patient access^{3,4}. While it has been widely demonstrated that mouse PSC are capable of robust expansion in scalable suspension bioreactors commonly used in industrial manufacturing processes with greater than 10 fold expansion in 4 days⁵⁻¹³, attempts to expand hPSC in these systems have been plagued by low maximum cell densities and low yields (<3.5×10⁶ cells/mL final cell density and <3 fold equivalent expansion in 4 days)¹⁴⁻¹⁹. These yields pose a significant technology gap to efficient translation of many large scale allogeneic hPSC-derived therapies as well as non-therapeutic applications. For example, it is estimated that allogeneic cardiomyocyte replacement therapy for heart disease, a condition affecting nearly 6 million adults in the United States, would require 10⁹ hPSC-derived cardiomyocytes per treatment⁴. Generating this number of cardiomyocytes from traditional hPSC cultures may require very large differentiation processes preceded by a seed train of progressively larger hPSC expansion bioreactors. Drug screening using hPSC also requires efficient production of large quantities of hPSC-derived cells to screen drug libraries.

As our understanding of the molecular basis of pluripotency deepens, there is mounting evidence suggesting that PSC exist in multiple states of pluripotency that can be manipulated by genetic engineering or by changing culture conditions²⁰. In vitro, mPSC exist in distinct states exhibiting unique epigenetic characteristics, self-renewal signal requirements, and differentiation potentials. Two of these states share similarities with stages of embryonic development: the “naïve” state resembles the pre-implantation inner cell mass and the primed state resembles the later, post-implantation epiblast, which is “primed” towards differentiation^{21,22}. The gene expression profile, epigenetic state, and signaling requirements of conventional hPSC resemble primed mPSC²⁰. Notably, mPSC have been cultured in conditions supporting the “naïve” state in all high yield, proof of principle bioreactor expansion experiments to date, while all hPSC bioreactor expansion studies to date have cultured cells in conditions supporting the conventional primed state.

Three main approaches have been taken to achieve a naïve-like state in human PSC: exogenous factor overexpression²³⁻²⁵, medium development based on biological understanding²⁶⁻²⁹, and systematic identification of medium components³⁰. Notable amongst these published reports is that many of these methods require conversion or maintenance of naïve PSC on MEF feeder layers. The unknown factors from MEF feeders required for stability lead to an unpredictable environment undesirable for scale-up and clinical use. Since initial descriptions of stabilization of pluripotency in mouse PSC³¹ by pharmacological blockage of ERK1/2 (the downstream target of FGF4), combined with activation of Wnt signaling (by inhibiting GSK3b), most naïve human PSC strategies have focused on ERK1/2 inhibition as an integral part of the medium development. Furthermore, many of the methods for human PSC require combinations of Activin and FGF, for which mouse and human embryo development has different requirements. Blocking ERK signaling via FGF inhibition suppresses hypoblast formation in mouse³² development but not human development³³, indicating a difference in the stabilizing effects of ERK inhibition between the two species in early embryonic development. These findings suggest either that further species differences exist between the cell states or that a stable, unique, and homogenous naïve human state has not yet been achieved.

Efforts to generate an alternative, high suspension yield hPSC state have been guided by recent reports on the generation of “naïve state” hpSC^{25,28-30,34,35}. Specifically, one report described a combination of 5 small molecule inhibitors that converts hPSC to an alternative state of pluripotency with properties including higher adherent single cell survival efficiencies and accelerated adherent growth rates³⁵. In this method, the hPSC were in an adherent cell culture system.

One method for scalable expansion of PSC is culture as cell aggregates in dynamic suspension conditions¹⁷. The PSC aggregate size distribution and average size is known to affect oxygen and nutrient diffusion throughout the aggregate and the resulting characteristics of the cell product. Lack of control over aggregate size can result in loss of cell viability and pluripotency. Polysulfated compounds, including heparin³⁶, polyvinyl sulfate³⁷, suramin³⁷, and dextran sulfate³⁸, have previously been used in the biopharmaceutical industry to maintain single-cell culture in bioreactors and prevent aggregation. Surprisingly, while others have used polysulfated compounds to prevent aggregation in the biopharmaceutical industry, the authors describe herein the use of controlled aggregation of pluripotent stem cells.

It is an object of the present disclosure to mitigate and/or obviate one or more of the above deficiencies.

### SUMMARY OF THE DISCLOSURE

In an aspect, a method for culturing pluripotent stem cells is provided. The method comprises a first step of culturing in a medium comprising inhibitors of GSK-3β, JNK, p38, PKC and Erk1/2, followed by a second step of culturing in a medium comprising inhibitors of GSK-3β, JNK, p38 and PKC, and not comprising an inhibitor of Erk1/2. In an embodiment, the first step of culturing is carried out under two dimensional culture conditions, and the second step is carried out under three dimensional culture conditions.

In an embodiment, the media do not contain serum.

In an embodiment, the medium of the second step further comprises dextran sulfate.

In a preferred embodiment, the pluripotent stem cells cultured in the second step are cultured as aggregates.

In a preferred embodiment, the pluripotent stem cells are human cells.

In an aspect, a method for generating a cell population from a population of pluripotent stem cells is provided. The method comprises a first step of culturing in a first medium comprising inhibitors of GSK-3β, JNK, p38, PKC and Erk1/2, followed by a second step of culturing in a medium comprising inhibitors of GSK-3β, JNK, p38 and PKC and not comprising inhibitors of Erk1/2, followed by a third step of culturing in a third medium comprising factors that induce cell differentiation. In an embodiment, the first step of culturing is carried out under two dimensional culture conditions, and the second and third steps are carried out under three dimensional culture conditions. In another embodiment, the first and third steps are carried out under two dimensional culture conditions, and the second step is carried out under three dimensional culture conditions.

In an embodiment, the media do not contain serum.

In a preferred embodiment, the pluripotent stem cells cultured under three dimensional conditions are cultured as aggregates.

In an aspect, a method for culturing pluripotent stem cell aggregates is provided. The method comprises culturing the cell aggregates in a medium containing a polysulfated compound.

In an embodiment, the pluripotent stem cell aggregates are cultured under three dimensional culture conditions.

In a preferred embodiment, the polysulfated compound is dextran sulfate.

In an aspect, a medium for the culture of pluripotent stem cells is provided. The medium comprises inhibitors of GSK-3β, JNK, p38 and PKC and does not comprise an inhibitor of Erk1/2.

In a preferred embodiment, the medium is serum-free.

In an embodiment, the medium further comprises fibroblast growth factor 2 (FGF2, bFGF), transforming growth factor-β1 (TGF-β1), a STAT3 activator, insulin, ascorbic acid, and albumin.

In an embodiment, the medium further comprises fibroblast growth factor 2 (FGF2, bFGF), transforming growth factor-β1 (TGF-β1), a STAT3 activator, insulin, ascorbic acid, albumin, N2 supplement, non-essential amino acids, glutamine, and serum replacement.

In an embodiment, the medium further comprises dextran sulfate.

In an aspect, a medium for the culture of pluripotent stem cells is provided. The medium comprises a polysulfated compound.

In a preferred embodiment, the polysulfated compound is dextran sulfate.

In an aspect, an isolated population of pluripotent stem cells generated by the method disclosed herein is provided.

In an embodiment, the isolated pluripotent stem cells are human.

### DESCRIPTION OF THE DRAWINGS

The patent or application file contains at least one drawing in color. Copies of this patent or patent application publication with color drawings will be provided by the Office upon request and payment of the necessary fee.

These and other features of the disclosure will become more apparent in the following detailed description in which reference is made to the appended drawings wherein:

FIGS. 1A-1E illustrate that a five inhibitor medium alters the morphology and gene expression of primed hPSC without impairing pluripotency.

FIG. 1A are images depicting that the characteristic morphology of 5i-hPSC colonies consists of rounded smooth borders and tightly packed cells, whereas primed hPSC colonies consist of less densely packed cells and do not display smooth rounded borders.

FIG. 1B is a graph showing that 5i-hPSC display a stable OCT4/SOX2 protein expression phenotype over multiple passages.

FIG. 1C depicts images of 5i-hPSC which are functionally pluripotent maintaining the same capacity to differentiate to cells of all three germ layers as control primed hPSC.

FIG. 1D is a graph depicting that 5i-hPSC have reduced expression of the CD24 surface marker used to track “naïve” state hPSC.

FIG. 1E is a histogram showing expression levels of pluripotency-related factors in adherent primed hPSC, adherent 5i-hPSC, and suspension 4i-hPSC normalized to the average primed HES2 GAPDH level. Significant differences exist between both adherent 5i- and suspension 4i-HES2 and primed conditions. (Tukey's HSD Test: *, **, and *** indicate p<0.05, 0.01, 0.001 respectively. Biological replicates: primed n=4, 5i-adherent n=9, 4i-suspension n=6. Error bars represent the standard deviation).

FIGS. 2A-G depict the characterization of bioprocess parameters responsible for enhanced manufacturability of hPSCs.

FIG. 2A is a graph depicting adherent growth curves for primed and 5i-hPSC (HES2 and H9 cell lines) on fibroblast feeder layers. Error bars represent the standard deviation.

FIG. 2B is a histogram showing that adherent doubling times of 5i-hPSC were significantly higher than primed hPSC (p<0.05, Student's T-Test, n=3. Error bars represent standard deviation.)

FIG. 2C is a graph showing that 5i-hPSC have a significantly higher single cell colony formation efficiency in suspension than primed cells in both HES2 and H9 cell lines (p<0.05, Kruskal-Wallis Test, n=4)

FIG. 2D is images showing that primed H9 and 5i-H9 cultured in static suspension conditions for 8 hours form aggregates and do not display accumulation of debris. 5i-H9 in dynamic suspension cultures display robust aggregate formation. In primed H9 dynamic suspension cultures, debris and large, dark non-viable cell clumps were observed.

FIG. 2E is images of 5i-HES2 cells and primed HES2 cells. In an orbital shaker suspension bioreactor after 72 hours of culture, 5i-HES2 form a larger number of smaller aggregates than primed HES2.

FIG. 2F is a graph depicting suspension growth curves of primed and 5i-hPSC. Error bars represent the standard deviation.

FIG. 2G is a graph depicting the maximum cell density reached by day 8 of culture in suspension. Cell densities reached in 5i conditions were significantly higher than in primed conditions (p<0.05, Tukey's HSD Test, n=4 5i-HES2, n=3 5i-H9 and primed HES2).

FIGS. 3A-E depict the identification of a new medium that enables long-term maintenance of a pluripotent state in high yield suspension culture.

FIG. 3A are graphs showing that loss of the OCT4/SOX2 positive pluripotent phenotype is observed over time in 5i-hPSC suspension cultures.

FIG. 3B is a graph showing that pluripotent marker expression levels are high in suspension 5i-hPSC for 3 days before 5i-hPSC levels decline over 9 days in suspension. Cell density peaks at day 8; however, pluripotent marker expression has already begun to decline.

### (Representative Data Shown)

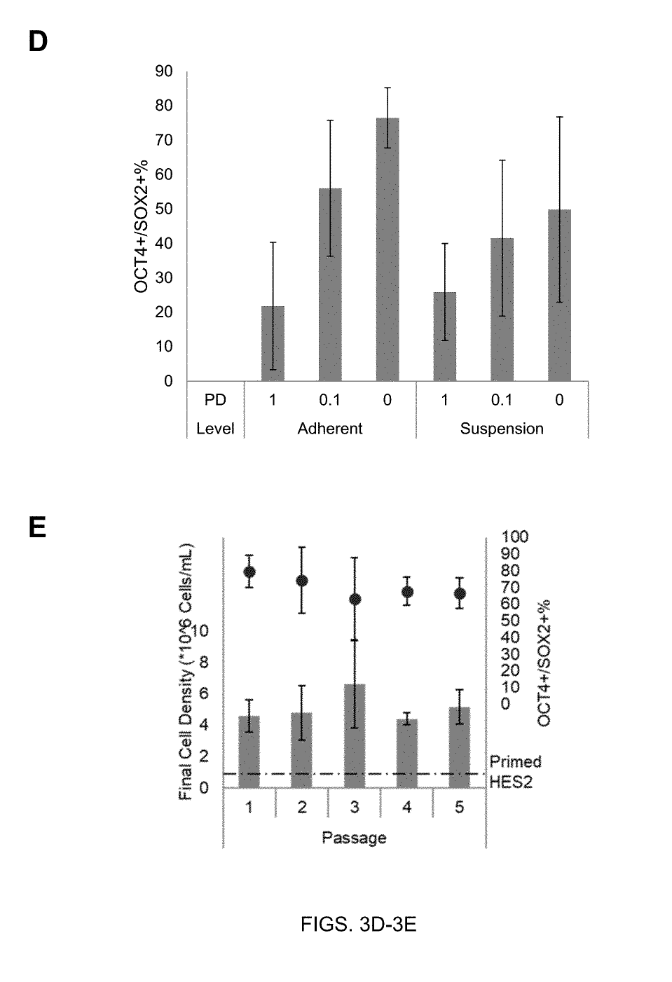

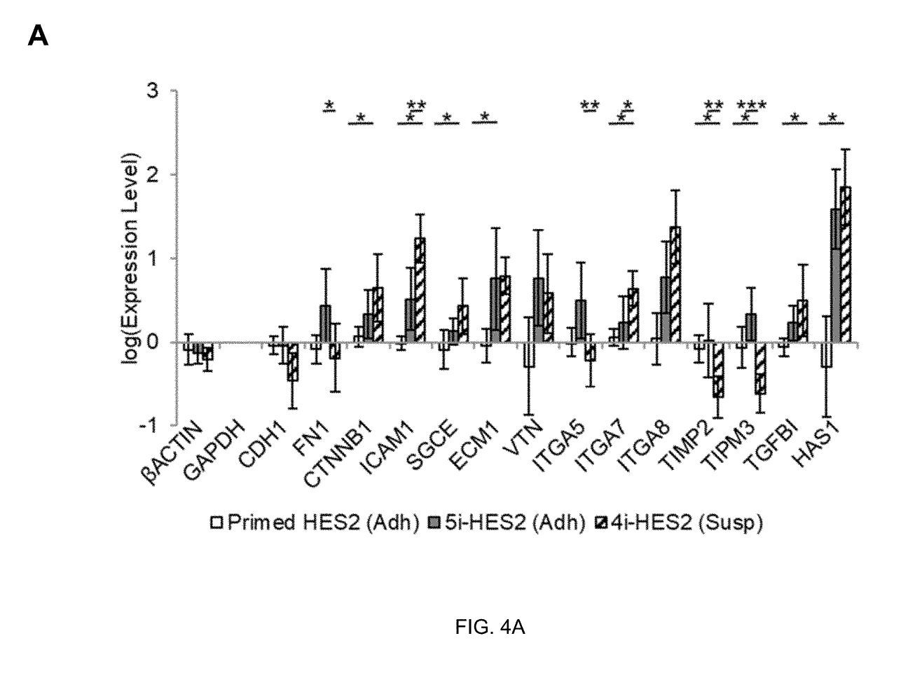

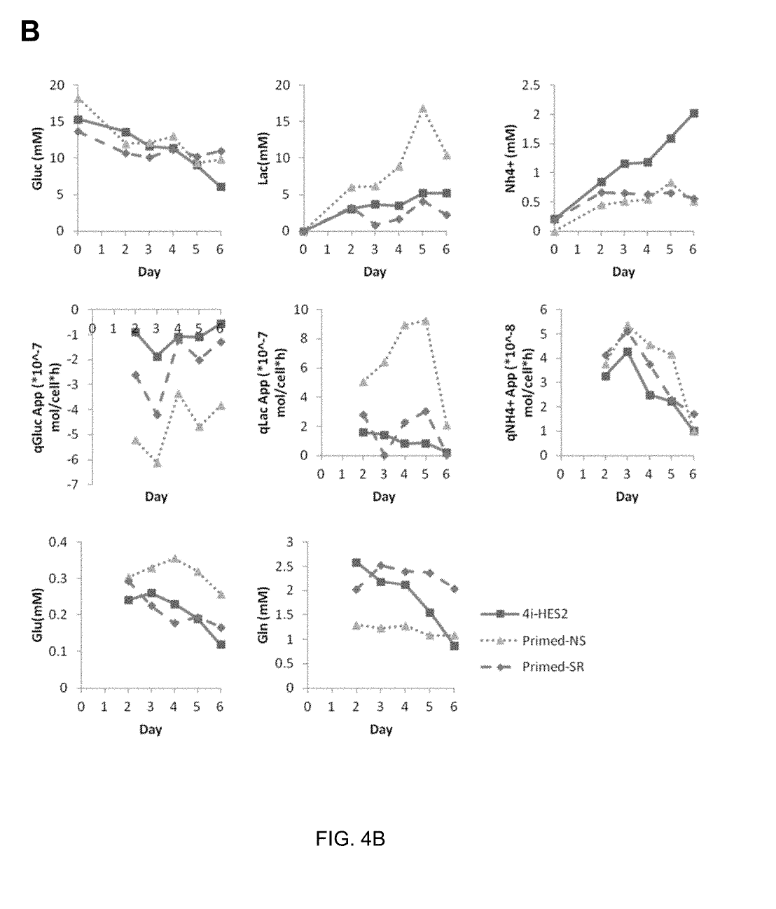

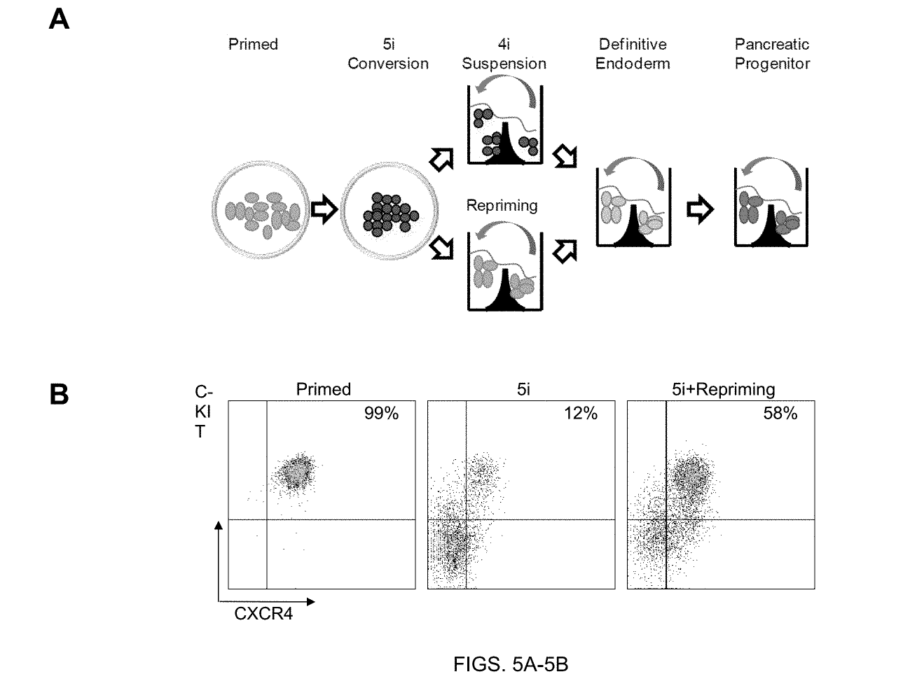

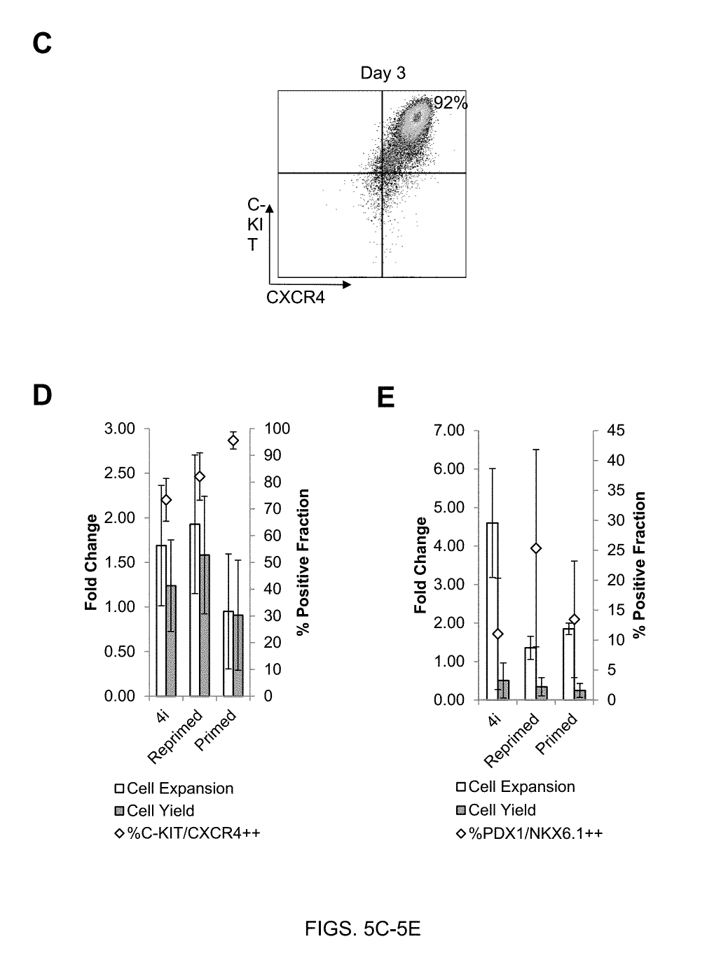

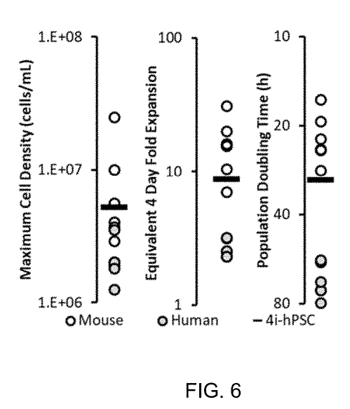

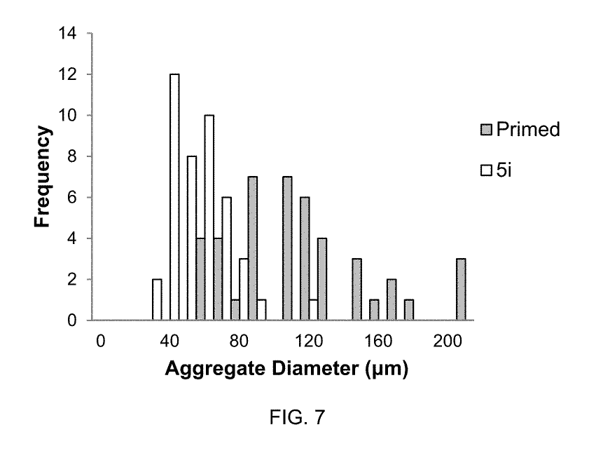

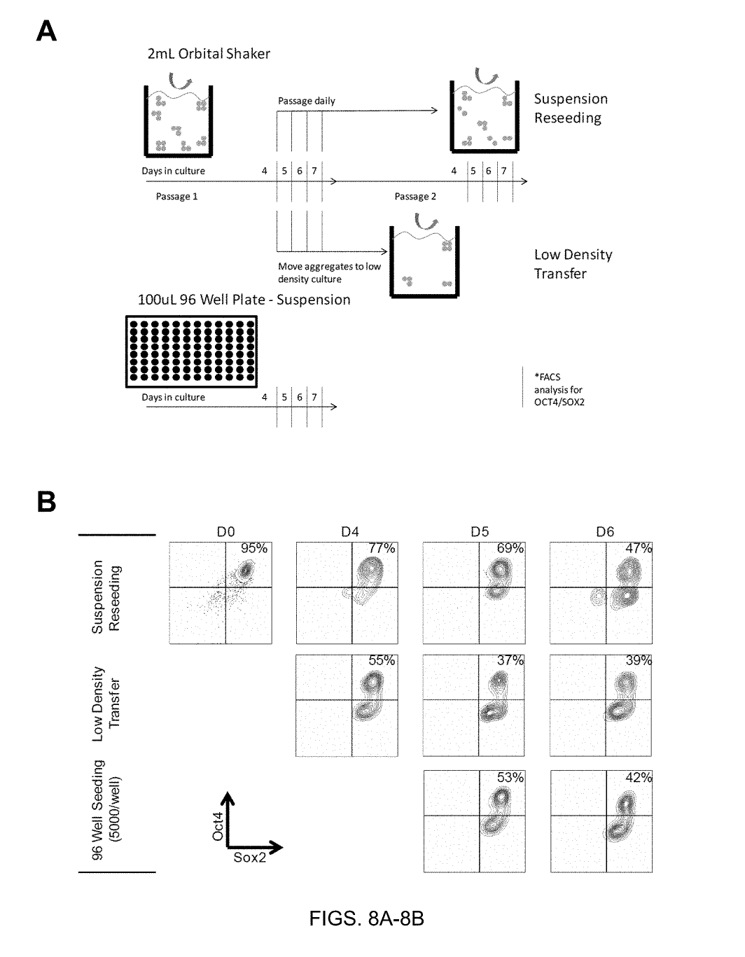

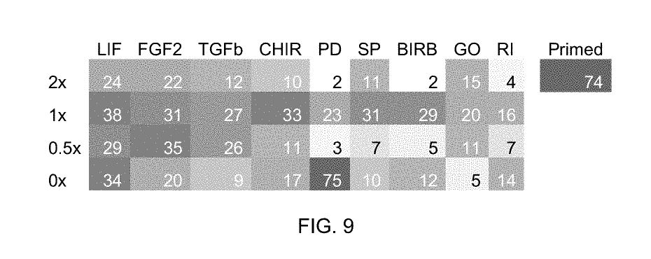

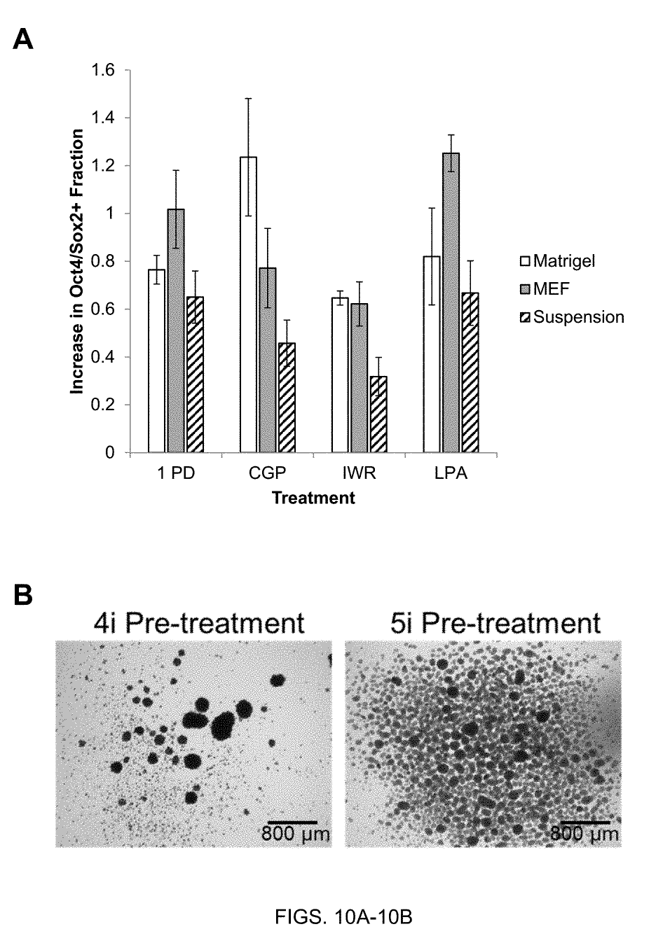

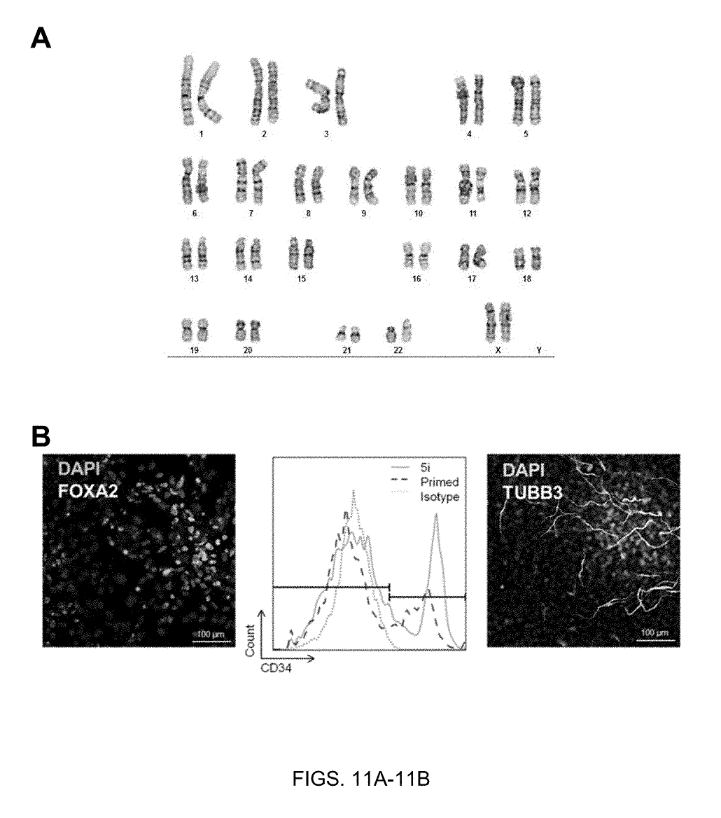

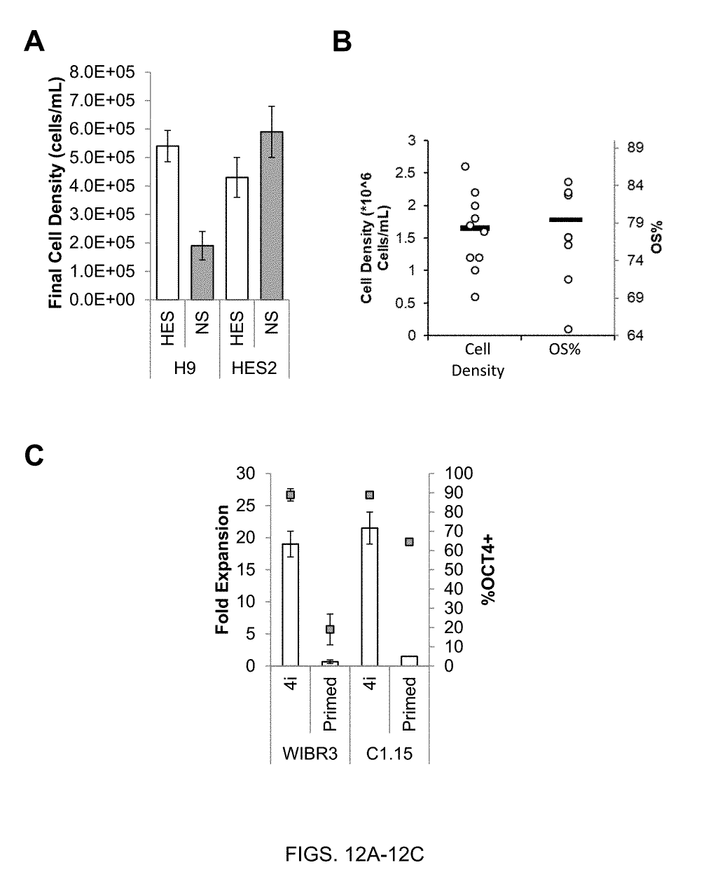

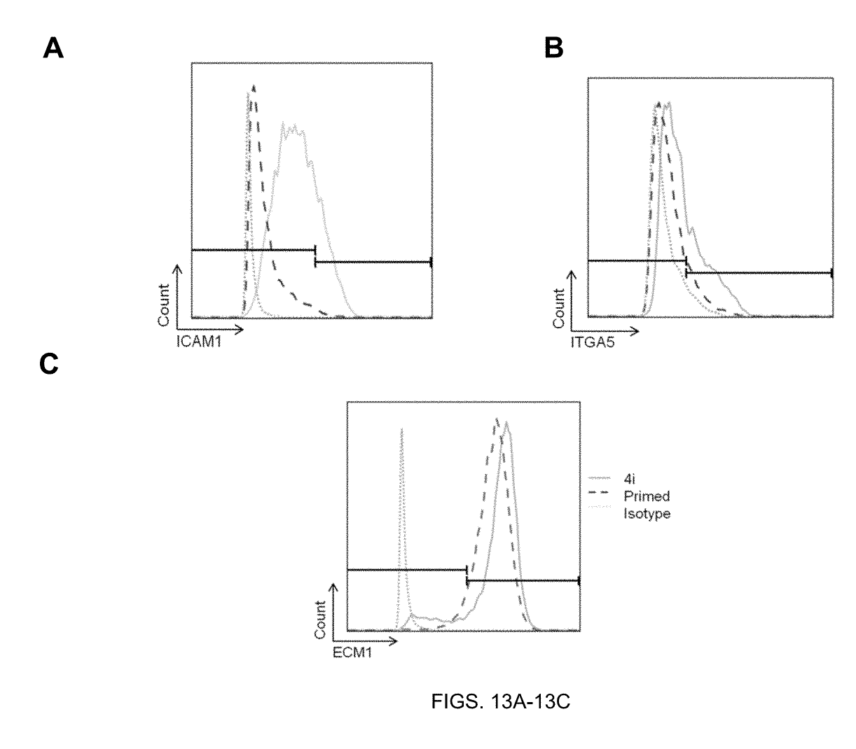

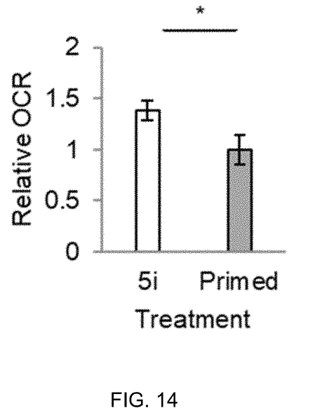

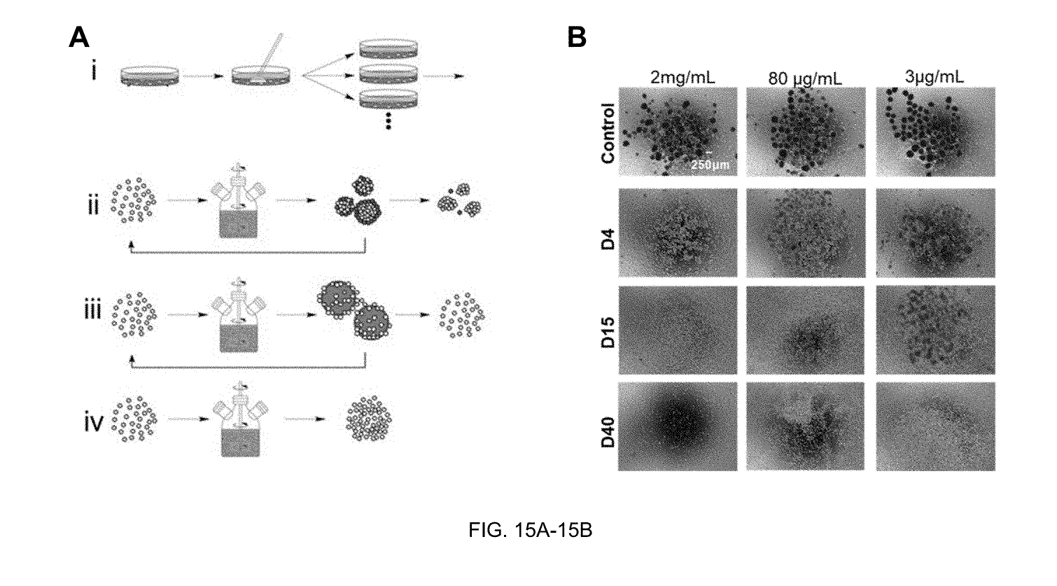

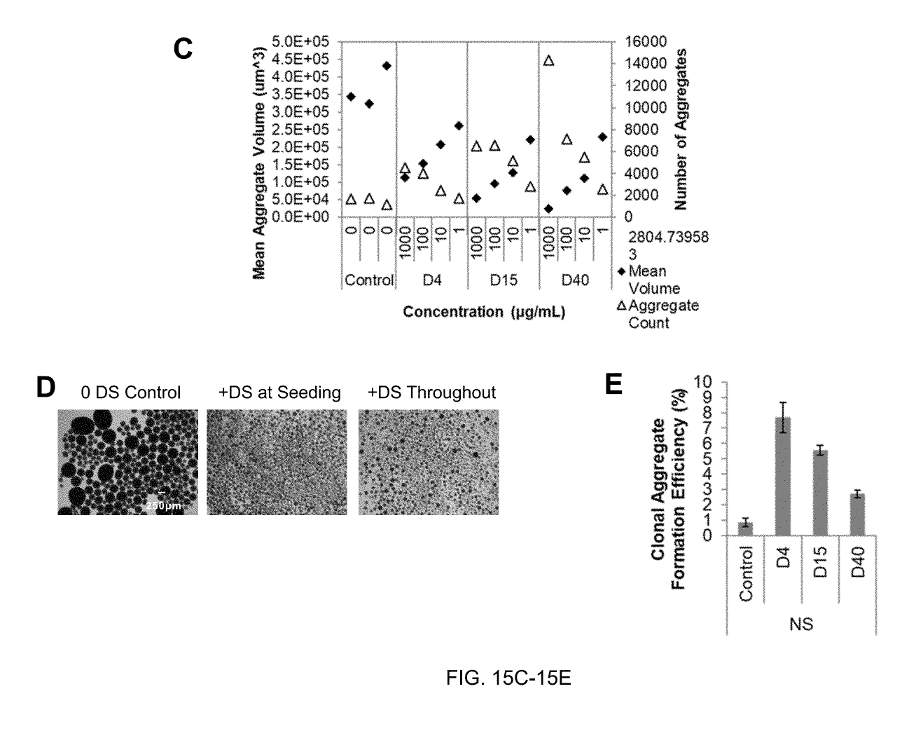

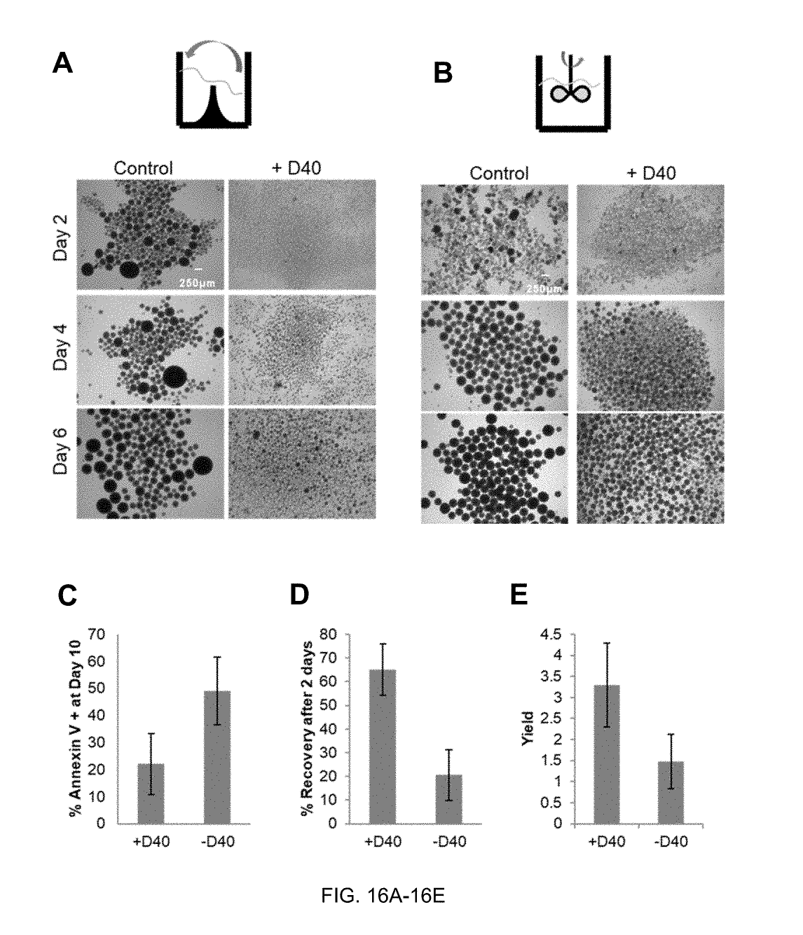

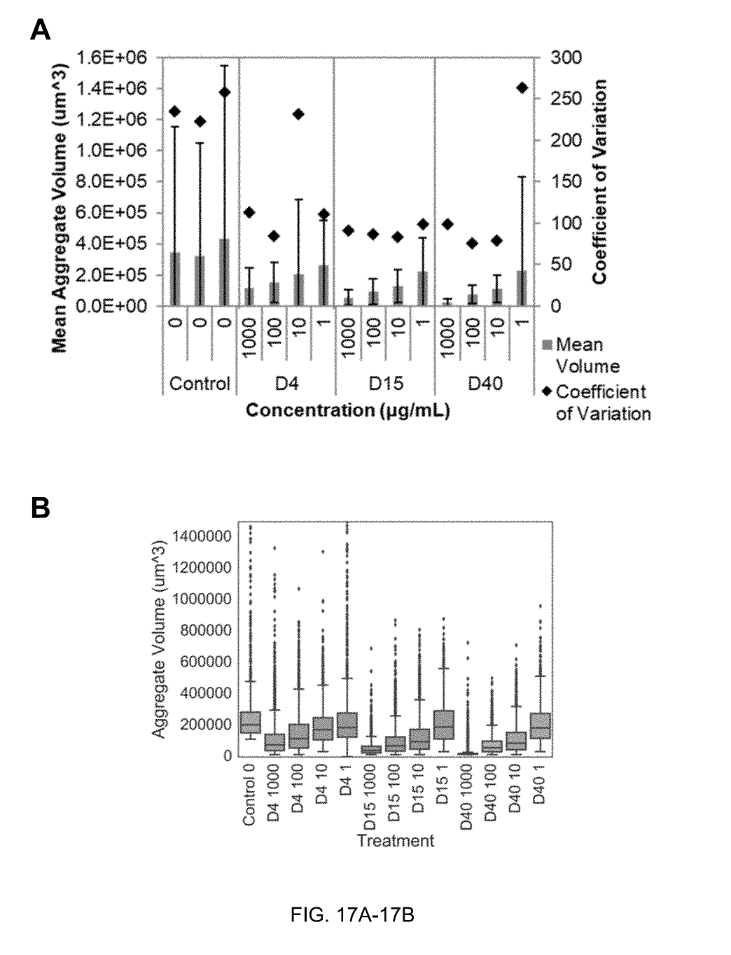

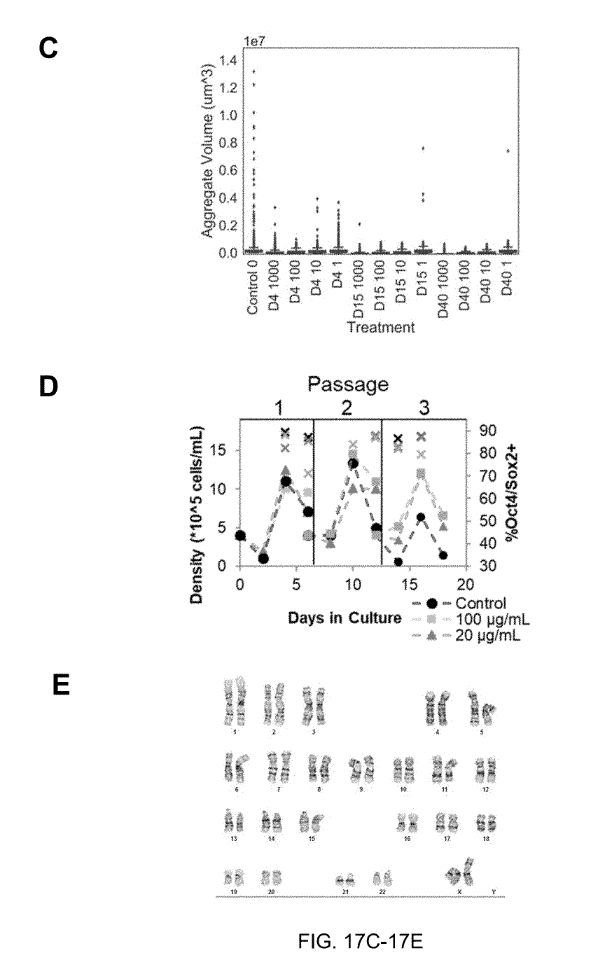

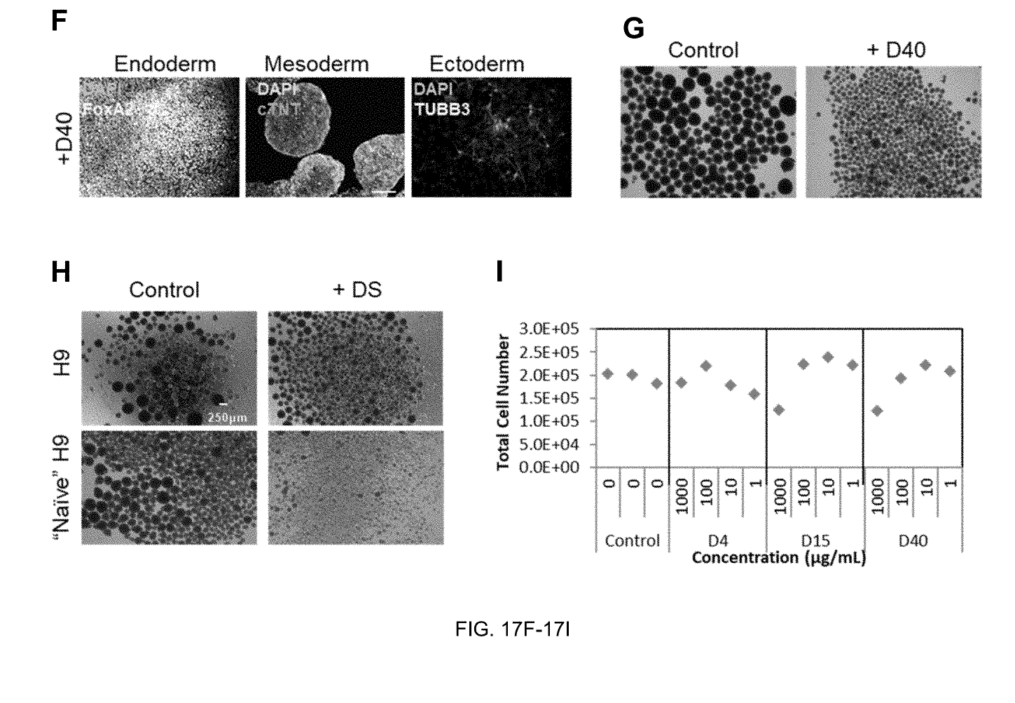

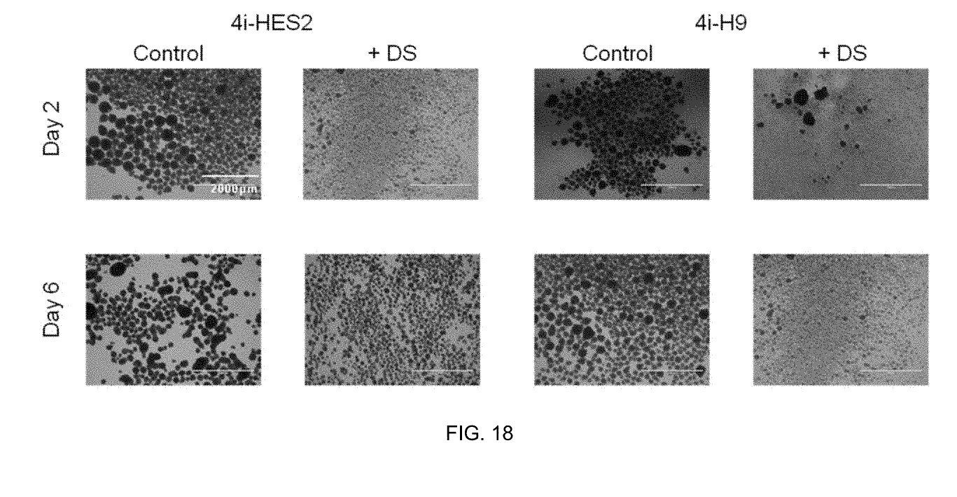

FIG. 3C is a graph showing that passaging 5i-hPSC at a day 6, a time point at which a high level of pluripotent marker expression is observed and the culture is still in exponential growth, did not enable maintenance of the pluripotent phenotype in the second passage. Error bars represent standard deviation.

FIG. 3D is a histogram showing that adding the PD inhibitor to feeder-free 5i-hPSC results in decreases in the OCT4/SOX2 pluripotent phenotype in both adherent and suspension conditions in a 24 well format. Error bars represent the standard deviation.

FIG. 3E is a graph showing that culturing HES2 in 5i medium without the PD inhibitor (4i)-HES2 for 5 passages resulted in maintenance of the pluripotent phenotype and cell densities in the range of 5×10⁶ cells/mL, higher than cell densities achieved for primed HES2 (dashed line) that averaged 6.7±0.2×10⁵ cells/mL. Error bars represent the standard deviation. Numbers of replicates for each of the 5 passages shown are (n=12,7,7,6,6) for final cell density and (n=14,9,4,3,3) for OCT4/SOX2%.

FIGS. 4A-B depict the characterization of suspension 4i-hPSC by distinct suspension adhesion molecule expression and medium utilization.

FIG. 4A is a histogram showing expression levels of select adhesion-related molecules in adherent primed hPSC, adherent 5i-hPSC, and suspension 4i-hPSC. Expression level is normalized to average primed HES2 GAPDH expression level. Significant differences exist between both adherent and suspension 5i-HES2 and primed conditions in select adhesion molecules as noted. (Tukey's HSD Test, *, **, and *** indicate p<0.05, 0.01, 0.001 respectively, biological replicates: primed n=4, 5i-adherent n=9, 4i-suspension n=6. Error bars represent standard deviation.)

FIG. 4B is graphs depicting level and metabolic rate of key metabolites in suspension expansion of hPSC. Primed hPSC are cultured in either NutriStem® (NS) or Serum Replacement (SR) medium, compared to 4i-hPSC. 4i-hPSC are observed to have reduced specific uptake and secretion of glucose and lactate as well as rapidly depleting glutamine and glutamate levels.

FIG. 5A-5E illustrate that 4i-hPSC can be directed to differentiate to pancreatic progenitors without repriming.

FIG. 5A is a schematic showing that differentiation of 5i-hPSC to pancreatic progenitors follows a 12-day directed differentiation protocol. Primed hPSC are converted to 4i-hPSC over several passages. 4i-hPSC are seeded into suspension and aggregate, followed by a 2 day re-priming stage. Definitive endoderm is induced between days 0-3 with Activin and CHIR followed by pancreatic progenitor specification at day 3-12. Days −2 to 0 are the “re-priming” stage in which 5i-hPSC aggregates are transferred to conventional primed hPSC medium.

FIG. 5B is graphs showing that differentiation of primed hPSC results in a high (>95%) percentage of definitive endoderm marked by c-kit/cxcr4 expression after 3 days in an adherent differentiation. 12% of differentiated 5i-hPSC express this phenotype at day 3 of differentiation, whereas 5i-hPSC re-primed in NutriStem® or Serum Replacement medium expressed this phenotype at 58% and 60% purity, respectively (Representative experiment shown).

FIG. 5C is a graph showing that in suspension optimized conditions, 4i-hPSC differentiate to a definitive endoderm phenotype at levels reaching 95% after 3 days

FIG. 5D is a histogram showing that 4i-hPSC and primed hPSC can differentiate to definitive endoderm progenitors with comparable cell yields and purities. No significant difference was observed at Day 3 in fold expansion and yield. Primed hPSC had significantly higher purity (p<0.005, Tukey's HSD). At day 12, no significant difference was observed in purity or yield, though 4i-hPSC had significantly higher fold expansion (p<0.01, Tukey's HSD). Error bars in this figure represent standard deviation.

FIG. 5E is a histogram showing that 4i-hPSC and primed hPSC can differentiate to pancreatic progenitors with comparable cell yields and purities. No significant difference was observed at Day 3 in fold expansion and yield. Primed hPSC had significantly higher purity (p<0.005, Tukey's HSD). At day 12, no significant difference was observed in purity or yield, though 4i-hPSC had significantly higher fold expansion (p<0.01, Tukey's HSD). Error bars in this figure represent standard deviation.

FIG. 6 illustrates that 4i-hPSC suspension expansion yields exceed published hPSC yields and approach mPSC yields. Maximum cell density, equivalent 4-day expansion, and suspension doubling times in the 4i-hPSC system are compared to published values for bioreactor cell expansion of mouse and human PSC⁵⁻¹⁹. Mouse and human PSC are compared to the results obtained in the 4i-hPSC.

FIG. 7 is a histogram depicting aggregate size distribution of primed hPSC and 5i-hPSC. After 3 days in orbital shaker (dynamic) suspension conditions, smaller aggregate sizes and size distribution were observed in in 5i treated cells in comparison to primed hPSC.

FIGS. 8A-B illustrate the effect of passage timing and cell density on loss of pluripotency in suspension.

FIG. 8A is a schematic of an experiment to determine if time of passaging or density at end of culture were responsible for loss of phenotype in 5i-hPSC. In this experiment, at days 4, 5, 6, and 7 of suspension expansion, aggregates were either dissociated (suspension reseeding) or separated for low density culture without dissociation (low density transfer). Schematic of 96 well plate setup for screening critical process parameters.

FIG. 8B is graphs depicting OCT4/SOX2 plots showing that dissociation followed by suspension reseeding as well as low density aggregate transfer did not rescue the pluripotent phenotype, regardless of timing. 96 well plate format recapitulates the results seen in 6 well plates.

FIG. 9 is a table depicting the effects of 5i medium components on the expression of the pluripotent phenotype (% OCT4/SOX2+). Each value in the table refers to the concentration of the corresponding component. One factor at a time screening strategy involved doubling, halving, or removing each component.

FIGS. 10A-B illustrate that additional pathway inhibition does not enable suspension culture in the presence of ERK inhibition.

FIG. 10A is a histogram showing that neither YAP/TAZ activation (LPA), SRC inhibition (CGP) nor Axin stabilization (IWR) increases the level of cells expressing the pluripotent phenotype in suspension. The OCT4/SOX2+ fraction is normalized to the OPD (4i) condition. Error bar represent standard deviation.

FIG. 10B is images of cells showing that primed hPSC treated with 5i in adherent conditions form a large number of small aggregates when cultured in suspension in 4i. Primed hPSC treated with 4i (no PD) in adherent conditions form a small number of large, dark aggregates in suspension in 4i.

FIGS. 11A-B depicts the further characterization of hPSC cultured in 5i medium in adherent conditions and 4i medium in suspension conditions.

FIG. 11A is an image depicting G-band karyotype of HES2 hPSC cultured for 5 passages in 5i in adherent conditions and 3 passages in 4i in suspension conditions. No karyotypic abnormalities are observed.

FIG. 11B is images of cells or a graph quantifying staining for endoderm (FOXA2), mesoderm (CD34), and ectoderm (TUBB3) markers in HES2 hPSC cultured for 5 passages in 5i in adherent conditions and 3 passages in 4i in suspension conditions.

FIGS. 12A-C depict the final cell density and phenotype of primed hPSC.

FIG. 12A is a histogram showing final cell density reached after 6 days of suspension culture of primed HES2 and H9 hPSC in Serum Replacement (HES) medium and Nutristem® (NS) medium. Error bars represent standard deviation.

FIG. 12B is a graph depicting final cell density and pluripotent phenotype (OS %: % OCT4+/SOX2+) of 4i-H9 in suspension culture.

FIG. 12C is a graph showing fold expansion after 6 days in suspension culture and % OCT4-GFP+ cells after 8 days in suspension culture of primed and 4i-treated WIBR3 hESC and C1.15 iPSC. Primed cultures are expanded in HES medium.

FIGS. 13A-C depict flow cytometry analysis of primed hPSCs, hPSCs in 4i medium, and isotype controls.

FIG. 13A depicts a flow cytometry plot for expression of ICAM1.

FIG. 13B depicts a flow cytometry plot for expression of ITGA5

FIG. 13C depicts a flow cytometry plot for expression of ECM1.

FIG. 14 is a histogram illustrating the oxygen consumption rate of adherent 5i and primed HES2 cells. Relative oxygen consumption rates (OCR) of adherent 5i and primed HES2, as measured by the MitoXpress® Xtra oxygen consumption rate assay. 5i OCR is normalized to Primed. * indicates p<0.05 by t-test. Error bars represent standard deviation.

FIGS. 15A-E depict hPSC aggregate size and formation efficiency with and without dextran sulfate.

FIG. 15A is a schematic showing that the adherent growth format (i) typically used in laboratory PSC culture are translated to suspension bioreactor technologies for industrialization. (ii) Suspension aggregate based culture can lead to heterogeneity in aggregation and serial passaging challenges. (iii) Microcarrier strategies are also used, and have unique challenges with serial passaging. (iv) Moving towards continuous single cell bioreactor expansion is an important future direction in the field.

FIG. 15B is images showing aggregate formation in orbital shaker suspension culture at different concentrations of dextran sulfate (DS) after 24 hours in the presence of rock inhibitor.

FIG. 15C is a graph depicting average aggregate volume and count in PSC suspension expansion after 4 days in culture (Representative experiment shown. Aggregate counts in the conditions shown from left to right are: 1654, 1741, 1182, 4515, 4023, 2412, 1722, 6510, 6587, 5195, 2823, 14303, 7182, 5516, and 2557).

FIG. 15D is images showing addition of dextran sulfate (40 kDa, D40) to suspension cultures at seeding only compared to daily addition.

FIG. 15E is a histogram illustrating aggregate formation efficiency of PSC seeded at clonal densities in suspension in Nutristem™ medium (n=4, p<0.05, t-test).

FIGS. 16A-E depict aggregation control and culture robustness with the addition of dextran sulfate.

FIG. 16A is images depicting PSC aggregates in orbital shaker suspension culture in the presence and absence of 100 μg/mL dextran sulfate (40 kDa, D40) at seeding.

FIG. 16B is images depicting PSC aggregates in stirred tank bioreactor culture in the presence and absence of 100 μg/mL dextran sulfate (40 kDa, D40) at seeding.

FIG. 16C is a histogram showing expression level of early apoptotic marker Annexin V after 10 days in orbital shaker culture (n=4, p<0.05, t-test).

FIG. 16D is a histogram showing cell recovery measured two days following seeding of passage 2 of PSC in suspension, in the presence and absence of 100 μg/mL dextran sulfate (n=4, p<0.05, t-test).

FIG. 16E is a histogram showing suspension cell yield in the presence and absence of 100 μg/mL dextran sulfate after two passages in suspension (n=6, p<0.05, t-test).

FIGS. 17A-171 illustrate the effects of dextran sulfate on hPSC aggregate volume, expansion, pluripotency and karyotype.

FIG. 17A is a graph showing aggregate volume and standard deviation in DS treated and untreated PSC cultured in suspension for 4 days. The coefficient of variation normalizes standard deviation to mean aggregate volume. Variances of treated conditions are all reduced relative to untreated conditions (2-sided F-test, p<0.0001) (Representative data shown).

FIG. 17B is a Box-and-whisker plot depicting aggregate volumes less than 1.5×10⁷ μm³ in DS treated and untreated PSC cultured in suspension for 4 days (Representative data shown).

FIG. 17C is a Box-and-whisker plot of aggregate volumes in DS treated and untreated PSC cultured in suspension for 4 days showing full range of data with outliers (Representative data shown).

FIG. 17D is a graph showing cell expansion and pluripotency marker expression, in control conditions and 20 and 100 μg/mL dextran sulfate (40 kDa, D40), over three passages in stirred suspension bioreactor culture showing expansion and maintenance of pluripotency marker expression (Oct4 and Sox2) (representative data shown).

FIG. 17E is images depicting karyotype analysis of PSC expanded in adherent conditions for 5 passages in the presence of DS followed by 4 passages in suspension in the presence of DS. No karyotypic abnormalities were noted.

FIG. 17F is images showing differentiation to all germ layers of PSC cultured in adherent conditions for 2 passages followed by expansion in the Ambr™ stirred tank bioreactor for 4 passages in the presence of 100 μg/mL dextran sulfate (40 kDa, D40). Staining for: pancreatic progenitors (endoderm, FoxA2 staining), cardiomyocytes (Troponin T staining), and neurons (β-3 Tubulin).

FIG. 17G is images showing PSC aggregates after 10 days in orbital suspension culture in the presence or absence of 100 μg/mL dextran sulfate (40 kDa, D40).

FIG. 17H is images showing H9 hPSC aggregates cultured in a medium containing DS in both conventional and “naïve” conditions after five days in suspension culture.

FIG. 17I is a graph depicting the total cell number of aggregates in FIG. 15C calculated by multiplying the total number of aggregates by the aggregate volume and dividing by an average cell volume calculated from a mean cell diameter of 17.5±0.4 (measured by MoxiZ™, Orflo®).

FIG. 18 is images of cells showing that culture of HES2 and H9 hPSCs in 4i suspension culture form a large number of small aggregates with the addition of dextran sulfate (DS) at day 2 and day 6 of culture.

### DETAILED DESCRIPTION OF THE DISCLOSURE

Unless defined otherwise, all technical and scientific terms used herein have the same meaning as commonly understood by one of ordinary skill in the art to which this disclosure belongs.

### Definitions

As used herein, the term “stem cell” refers to a cell that can differentiate into more specialized cells and has the capacity for self-renewal. Stem cells include pluripotent stem cells (PSCs), such as embryonic stem cells (ESCs) and induced pluripotent stem cells (iPSCs), and multipotent stem cells, such as cord blood stem cells, and adult stem cells, which are found in various tissues.

As used herein, a “defined culture medium” refers to a chemically-defined formulation comprised solely of chemically-defined constituents. A defined medium may include constituents having known chemical compositions. Medium constituents may be synthetic and/or derived from known non-synthetic sources. For example, a defined medium may include one or more growth factors secreted from known tissues or cells. However, the defined medium does not include the conditioned medium from a culture of such cells. A defined medium may include specific, known serum components isolated from an animal, including human serum components, but the defined medium does not include serum. Any serum components provided in the defined medium such as, for example, bovine serum albumin (BSA), are preferably substantially homogeneous.

As used herein, “serum-free medium” refers to a cell culture medium that lacks animal serum. Serum-free medium may include specific, known serum components isolated from an animal (including human animals), such as, for example, BSA.

As used herein, the term “GSK-3β inhibitor” refers to any molecule that inhibits the activity of glycogen synthase kinase 3 beta (GSK-3β). Non-limiting examples of GSK-3β inhibitors include 6-[[2-[[4-(2,4-dichlorophenyl)-5-(5-methyl-1H-imidazol-2-yl)-2-pyrimidinyl]amino]ethyl]amino]-3-pyridinecarbonitrile (CHIR99021), (2′Z,3′E)-6-bromoindirubin-3′-oxime (BIO), N-[(4-Methoxyphenyl)methyl]-N′-(5-nitro-2-thiazolyl)urea (AR-A 014418), 9-bromo-7,12-dihydro-indolo[3,2-d][1]benzazepin-6(5H)-one (Kenpaullone), dichlorophenyl)-4-(1-methyl-1H-indol-3-yl)-1H-pyrrole-2,5-dione (SB 216763), and 3-[(3-chloro-4-hydroxyphenyl)amino]-4-(2-nitrophenyl)-1H-pyrrole-2,5-dione (SB 415286). Methods for assessing the activity of GSK-3β are known to those skilled in the art to which this disclosure relates.

As used herein, the term “JNK inhibitor” refers to any molecule that inhibits the activity of c-Jun N-terminal kinase (JNK). Non-limiting examples of JNK inhibitors include anthra[1-9-cd]pyrazol-6(2H)-one (SP 600125), 4-(2,3-dihydro-1,4-benzodioxin-6-yl)-2,4-dihydro-5-[(5-nitro-2-thiazolyl)thio]-3H-1,2,4-triazol-3-one (BI 78D3), and 5-[(5-nitro-2-thiazolyl)thio]-1,3,4thiadiazol-2-amine (SU 3327). Methods for assessing the activity of JNK are known to those skilled in the art to which this disclosure relates.

As used herein, the term “p38 inhibitor” refers to any molecule that inhibits the function of p38 mitogen-activated protein kinase (MAPK) family members (p38). Non-limiting examples of p38 inhibitors include 1-(5-tert-Butyl-2-p-tolyl-2H-pyrazol-3-yl)-3-[4-(2-morpholin-4-yl-ethoxy)naphthalen-1-yl]urea (BIRB796), 4-[5-(4-Fluorophenyl)-2-[4-(methylsulfonyl)phenyl]-1H-imidazol-4-yl]pyridine (SB 203580), trans-4-[4-(4-Fluorophenyl)-5-(2-methoxy-4-pyrimidinyl)-1H-imidazol-1-yl]cyclohexanol (SB 239063), 4-[4-(4-Fluorophenyl)-5-(4-pyridinyl)-1H-imidazol-2-yl]phenol (SB 202190), and 2,2-Sulfonyl-bis-(3,4,6-trichlorophenol) (p38 MAP Kinase Inhibitor IV). Methods for assessing the function of p38 mitogen-activated protein kinase (MAPK) family members (p38) are known to those skilled in the art to which this disclosure relates.

As used herein, the term “PKC inhibitor” refers to any molecule that inhibits the function of protein kinase C (PKC). Non-limiting examples of PKC inhibitors include 3-[1-[3-(Dimethylamino)propyl]-5-methoxy-1H-indol-3-yl]-4-(1H-indol-3-yl)-1H-pyrrole-2,5-dione (Go 6983), and (9S,10R,12R)-2,3,9,10,11,12-hexahydro-10-hydroxy-9-methyl-1-oxo-9,12-epoxy-1H-diindolo[1,2,3-fg:3′,2′,1′-kl]pyrrolo[3,4-i][1,6]benzodiazocine-10-carboxylic acid methyl ester (K252a). Methods for assessing the function of PKC are known to those skilled in the art to which this disclosure relates.

As used herein, the term “Erk1/2 inhibitor” refers to any molecule that inhibits the function of extracellular-signal related kinase 1 and/or extracellular-signal related kinase 2 (Erk1/2). Non-limiting examples of Erk1/2 inhibitors include N-[(2R)-2,3-dihydroxypropoxy]-3,4-difluoro-2-[(2-fluoro-4-iodophenyl)amino]-benzamide (PD 0325901), 2′-amino-3′-methoxyflavone (PD 98059), and 2-(2-chloro-4-iodophenylamino)-N-cyclopropylmethoxy-3,4-difluorobenzamide (PD 184352). In an embodiment, the Erk1/2 inhibitor inhibits Erk1 function. In an embodiment the Erk1/2 inhibitor inhibits Erk2 function. In an embodiment, the ERK1/2 inhibitor inhibits both Erk1 and Erk2 function. Methods for assessing the function of Erk1 and Erk2 are known to those skilled in the art to which this disclosure relates.

As used herein, the term “STAT3 activator” refers to any molecule that activates the transcription factor signal transducer and activator of transcription 3 (STAT3). Non-limiting examples of STAT3 activators include interferon, epidermal growth factor (EGF), interleukin 5 (IL-5), interleukin 6 (IL-6), hepatocyte growth factor (HGF), bone morphogenetic protein 2 (BMP2) and leukemia inhibitory factor (LIF). Methods for assessing the function of STAT3 are known to those skilled in the art to which this disclosure relates.

As used herein, the term “polysulfated compound” refers to any molecule with multiple sulfate moieties. Non-limiting examples of polysulfated compounds include dextran sulfate, heparin, polyvinyl sulfate, pentosan polysulfate, and suramin.

As used herein, the term “static culture” refers to cell culture that is carried out without agitation of the culture medium. Non-limiting examples of static culture include adherent culture in a multi-well plate without agitation and cell culture in a matrix or hydrogel without fluid flow or agitation. An “adherent culture” is an example of a static culture system, wherein cells are cultured in two-dimensions. The cells may be cultured and/or differentiated under two dimensional conditions, for example, by culturing and/or differentiating them on the bottom of wells of tissue culture plates or the bottoms of tissue culture flask. In an embodiment, the bottoms of tissue culture plates wells or flasks may be coated with an extracellular matrix protein(s) or a hydrogel, and the cells may grow and/or differentiate on the extracellular matrix protein(s) or hydrogel in a two dimensional manner.

As used herein, the term “dynamic culture” refers to cell culture that is carried out with agitation of the culture medium. Non-limiting examples of dynamic culture include culture of cell aggregates in a culture system on an orbital shaker, culture of cells in a stirred suspension bioreactor, and culture of cells in a perfusion bioreactor.

The cells may be cultured and/or differentiated under three dimensional conditions, for example, by culturing and/or differentiating them as single cell suspensions, as multicellular aggregates, on microcarriers or in/on extracellular matrix protein(s) or a hydrogel in tissue culture plates, tissue culture flasks, or in bioreactors.

### GENERAL DESCRIPTION OF THE DISCLOSURE

As described herein, the inventors have determined an in vitro method for culturing pluripotent stem cells. The method involves a first step of culturing pluripotent stem cells in a first medium comprising inhibitors of GSK-3β, JNK, p38, PKC and Erk1/2 (also referred to herein as “5i medium”), followed by a second step of culturing in a second medium comprising inhibitors of GSK-3β, JNK, p38 and PKC, and lacking an inhibitor of Erk1/2 (also referred to herein as “4i medium”). In an embodiment, the inventors found that sequential culture in the two media resulted in enhanced pluripotent stem cell proliferation and yield. In various embodiments, the method can be used to generate pluripotent stem cells in suspension culture.

For example, pluripotent stem cells may be initially cultured in the first medium comprising inhibitors of GSK-3β, JNK, p38, PKC and Erk1/2 in adherent culture, and then in the second medium comprising inhibitors of GSK-3β, JNK, p38 and PKC, and lacking an inhibitor of Erk1/2, in dynamic suspension culture.

As described herein, the inventors have developed a medium for the culture of pluripotent stem cells. The medium comprises inhibitors of GSK-3β, JNK, p38 and PKC, and lacks inhibitors of Erk1/2.

As described herein, the inventors have developed an in vitro method for the culture of pluripotent stem cell aggregates. The method comprises culture in a medium comprising a polysulfated compound.

In an embodiment, pluripotent stem cells may be cultured as aggregates under dynamic suspension conditions in a medium comprising the polysulfated compound dextran sulfate, salts, and derivatives thereof. In a preferred embodiment, the dextran sulfate has a Chemical Abstract Service number of 9011-18-1.

In an embodiment, the dextran sulfate has a molecular weight of about 4.000 Da to about 500,000 Da.

In an embodiment, the dextran sulfate is a salt. In a preferred embodiment, it is a sodium salt.

In an embodiment, the dextran sulfate has a sulfate content of about 2% to about 20%.

As described herein, the inventors have developed a medium for the culture of pluripotent stem cell aggregates. The medium comprises a polysulfated compound. In an embodiment, the polysulfated compound is dextran sulfate.

Pluripotent stem cells generated using the methods provided herein are provided. The cells provided herein may be used, for example, to generate a differentiated cell population, as described further below.

Method of Culturing Pluripotent Stem Cells

Generally, the in vitro method of culturing pluripotent stem cells involves a first step of culturing the cells in a first medium comprising one or more inhibitors of each of GSK-3β, JNK, p38, PKC and Erk1/2, followed by a second step of culturing in a second medium comprising one or more inhibitors of GSK-3β, JNK, p38 and PKC, and lacking an inhibitor of Erk1/2. To confirm maintenance of pluripotency, the cells can be analyzed for one or more features indicative of pluripotent stem cells, such as, for example, one or more phenotypic cell markers known to those skilled in the art to which this disclosure relates, such as OCT4 and/or SOX2. Methods for assessing pluripotency are known to those skilled in the art.

In an embodiment, the pluripotent stem cells are ESCs or iPSCs. In a preferred embodiment, the pluripotent stem cells are human cells.

In an embodiment, the first step of the method is performed in adherent culture, and the second step is performed in dynamic suspension culture.

In an embodiment, the pluripotent stem cells cultured in dynamic suspension culture are cultured as aggregates.

In an embodiment, preferred culture vessels for culture of pluripotent stem cells in adherent and dynamic suspension conditions are provided.

In an embodiment, the media provided in the first and second steps of the method further comprise one or more of fibroblast growth factor 2 (FGF2, bFGF), transforming growth factor-β1 (TGF-β1), a STAT3 activator (such as, for example, leukemia inhibitory factor [LIF]), insulin, ascorbic acid, albumin, N2 supplement, non-essential amino acids, glutamine (provided, for example, by L-glutamine or L-alanyl-L-glutamine dipeptide), and serum replacement. In an embodiment, the media further comprises all of the above molecules.

In an embodiment, the medium provided in the second step of the method further comprises dextran sulfate.

In an embodiment, the media provided in the first and second steps of the method are defined.

In an embodiment, human pluripotent stem cells are cultured on feeder layers of irradiated mouse embryonic fibroblasts in a medium comprising the GSK-3β inhibitor CHIR99021, the JNK inhibitor SP600125, the p38 inhibitor BIRB796, the PKC inhibitor Go6983, and the Erk1/2 inhibitor PD0325901. In a preferred embodiment, the medium further comprises 2% serum replacement, glutamine (provided, for example, by L-glutamine or L-alanyl-L-glutamine dipeptide), non-essential amino acids supplement, N2 supplement, insulin, ascorbic acid, a STAT3 activator (such as, for example, leukemia inhibitory factor [LIF]), TGFβ1 and a basal medium such as, for example, DMEM/F12. After 5-8 passages, the human pluripotent stem cells are dissociated into single cells and seeded into a bioreactor at an appropriate density, e.g., 2×10⁵ cells/mL, in a medium comprising the GSK-3β inhibitor CHIR99021, the JNK inhibitor SP600125, the p38 inhibitor BIRB796, the PKC inhibitor Go6983, but not comprising the Erk1/2 inhibitor PD0325901. In a preferred embodiment, the medium further comprises 2% serum replacement, glutamine (provided, for example, by L-glutamine or L-alanyl-L-glutamine dipeptide), non-essential amino acids supplement, N2 supplement, insulin, ascorbic acid, a STAT3 activator (such as, for example, leukemia inhibitory factor [LIF]), TGFβ1 and a basal medium such as, for example, DMEM/F12. Medium exchange is performed as appropriate, e.g. a 50% medium exchange daily beginning 2 days after seeding. The human pluripotent stem cells can be passaged by dissociation into single cells and re-seeding into new media at appropriate intervals, e.g. every 5-6 days. To confirm maintenance of pluripotency, the cells cultured in the disclosed medium can be analyzed for one or more features indicative of pluripotent stem cells, such as, for example, specific molecular markers known to those skilled in the art to which this disclosure relates, such as OCT4 and/or SOX2. Methods for assessing pluripotency are known to those skilled in the art.

In an aspect, the pluripotent stem cells cultured in the first and second media are then differentiated by culturing under conditions that induce differentiation to a specific cell type(s). In an embodiment, the pluripotent stem cells are differentiated towards a progenitor cell type such as, for example, cardiac progenitor cells, pancreatic progenitor cells, neural progenitor cells or hematopoietic stem/progenitor cells. In an embodiment, the pluripotent stem cells are differentiated to cardiac cells.

Media for Culture of Pluripotent Stem Cells

In an aspect, a medium for inducing proliferation of pluripotent stem cells is provided. For example, in an embodiment, the medium comprises one or more inhibitors of GSK-3β, JNK, p38 and PKC, and does not comprise an inhibitor of Erk1/2. In a preferred embodiment, the medium further comprises one or more of fibroblast growth factor 2 (FGF2, bFGF), transforming growth factor-β1 (TGF-β1), a STAT3 activator (such as, for example, leukemia inhibitory factor [LIF]), insulin, ascorbic acid, albumin, N2 supplement, non-essential amino acids, glutamine (provided, for example, by L-glutamine or L-alanyl-L-glutamine dipeptide), and serum replacement. In an embodiment, the medium further comprises all of the above molecules.

In an embodiment, the medium further comprises dextran sulfate.

In an embodiment, described further herein below, it is contemplated that the media provided herein may be used to generate a population of pluripotent stem cells. In a preferred embodiment, the population of pluripotent stem cells are cultured as aggregates in dynamic suspension.

In an embodiment, it is contemplated that the media provided herein may be used to generate pluripotent stem cells that are then differentiated into a cell type of interest. For example, it may be desired to generate a population of pancreatic progenitor cells. Following the expansion of the pluripotent stem cells in the disclosed media, the pluripotent stem cells are then cultured in a media that directs their differentiation to pancreatic progenitors. In a preferred embodiment, this media allows for an enhanced yield of cells. Further yield enhancement can be attributed to more consistent cell compositions in the cultures, and enhanced cell numbers.

Method for Culture of Pluripotent Stem Cell Aggregates

In an aspect, a method for culture of pluripotent stem cell aggregates is provided. The method comprises culture of the cell aggregates in a medium comprising a polysulfated compound. The polysulfated compound enables the culture of homogeneous cell aggregates. Methods of analyzing aggregate size are known to those skilled in the art.

In an embodiment, the mean aggregate volume of pluripotent stem cells cultured in a medium comprising a polysulfated compound is about 0.1×10⁵ cubic micrometers to about 3×10⁵ cubic micrometers.

In an embodiment, pluripotent stem cells cultured as aggregates in a medium comprising a polysulfated compound grow as aggregates with a coefficient of variation in aggregate size that is smaller than pluripotent stem cells cultured as aggregates in a medium without a polysulfated compound. In a preferred embodiment, the coefficent of variation of the aggregate volume of pluripotent stem cells cultured in a medium comprising a polysulfated compound is more than 50% lower than the coefficient of variation of aggregate volume of pluripotent stem cells cultured in a medium not comprising a polysulfated compound.

In an embodiment, the coefficient of variation of pluripotent stem cells cultured in a medium comprising a polysulfated compound is about 25% to about 150%.

In an embodiment, pluripotent stem cells cultured as aggregates in a medium comprising a polysulfated compound result in fewer large aggregate size outliers than pluripotent stem cells cultured as aggregates in a medium without a polysulfated compound.

In an embodiment, pluripotent stem cells cultured as aggregates in a medium comprising a polysulfated compound grow as smaller aggregates when they are cultured in a medium comprising a higher concentration of a polysulfated compound than a medium comprising a lower concentration of a polysulfated compound.

In an embodiment, pluripotent stem cells cultured as aggregates in a medium comprising a polysulfated compound grow as smaller aggregates when they are cultured in a medium comprising a polysulfated compound with higher molecular weight than a medium comprising a polysulfated compound with lower molecular weight.

In an embodiment, the pluripotent stem cells are cultured in a medium comprising the polysulfated compound dextran sulfate. The other components of the medium may be a medium formulation suitable for pluripotent stem cell culture such as, for example, Nutristem™ medium or E8™ medium.

In an embodiment, the pluripotent stem cell aggregates cultured in a medium comprising a polysulfated compound are cultured under dynamic suspension conditions. Dynamic suspension conditions may comprise, for example, culture on an orbital shaker or culture in a stirred tank bioreactor (STR). The pluripotent stem cell aggregates may be cultured over multiple passages, such as, for example, for up to 9 passages.

In an aspect, the pluripotent stem cell aggregates cultured in a medium comprising a polysulfated compound are then differentiated by culturing under conditions that induce differentiation to a specific cell type(s). In an embodiment, the pluripotent stem cells are differentiated towards a progenitor cell type such as, for example, pancreatic progenitor cells. In an embodiment, the pluripotent stem cells are differentiated to cardiomyocytes. In an embodiment, the pluripotent stem cells are differentiated to neurons.

Media for Culture of Pluripotent Stem Cell Aggregates

In an aspect, a medium for the culture of pluripotent stem cell aggregates is provided. The medium comprises a polysulfated compound.

In an embodiment, the polysulfated compound is dextran sulfate.

In an embodiment, it is contemplated that the media provided herein may be used to generate a population of pluripotent stem cells. In a preferred embodiment, the population of pluripotent stem cells are generated under dynamic suspension conditions. In a preferred embodiment, the pluripotent stem cell aggregates are substantially homogenous in size.

Pluripotent Stem Cells Generated Using the Media and Method Provided Herein

Pluripotent stem cells generated using the media and method provided herein are provided. In an embodiment, the pluripotent stem cells can be characterized genotypically via expression of pluripotent markers such as, for example, Oct4 and SOX2. In an embodiment, the pluripotent stem cells provided herein may be characterized by an increased growth rate. In an embodiment, the pluripotent stem cells provided herein may be characterized by a smaller and/or more uniform aggregate size distribution.

In an embodiment, the pluripotent stem cells are ESCs or iPSCs. In a preferred embodiment, the pluripotent stem cells are human cells.

In an embodiment, the pluripotent stem cells generated using the method provided herein are autologous.

In an embodiment, the pluripotent stem cells generated using the method provided herein are allogeneic.

The cells provided herein may be used, for example, to provide a cell source for developing cell-based therapies to cure a variety of chronic conditions. The methods and reagents provided in this disclosure offer robust and scalable technologies for manufacturing the quantities of cells anticipated to be required for widespread patient access. The methods and reagents provided in this disclosure show that hPSC are capable of expansion in scalable suspension bioreactors commonly used in industrial manufacturing processes with traditional hPSC cultures. In particular the hPSC produced according to the method of the present disclosure are distinct from traditional hPSC cultures in suspension expansion yield, intensification (i.e. cell density), and growth rates. The methods and reagents provided in this disclosure are particularly well suited to large scale allogeneic hPSC-derived therapies, such as allogeneic cardiomyocyte replacement therapy for heart disease, and for drug library screening techniques using hPSC.

Kits for Culturing Pluripotent Stem Cells

The present disclosure contemplates kits for carrying out the methods provided herein. Such kits typically comprise two or more components required for culture of pluripotent stem cells. Components of the kit include, but are not limited to, one or more of compounds, reagents, containers, equipment and instructions for using the kit. Accordingly, the methods described herein may be performed by utilizing pre-packaged kits provided herein.

In an embodiment, a kit for use to expand pluripotent stem cells in vitro is provided. The kit comprises one or more inhibitors of each of GSK-3β(such as, for example, CHIR99021), JNK (such as, for example, SP600125), p38 (such as, for example, BIRB796), PKC (such as, for example, Go6983), and Erk1/2 (such as, for example, PD0325901). In a preferred embodiment, the kit further comprises one or more of fibroblast growth factor 2 (FGF2, bFGF), transforming growth factor-β1 (TGF-β1), a STAT3 activator (such as, for example, leukemia inhibitory factor [LIF]), insulin, ascorbic acid, albumin, N2 supplement, non-essential amino acids, glutamine (provided, for example, by L-glutamine or L-alanyl-L-glutamine dipeptide), and serum replacement. In an embodiment, the kit further comprises a basal medium (such as, for example, DMEM/F12). In an embodiment, the kit further comprises all of the above molecules.

In an embodiment, a kit for use to culture pluripotent stem cell aggregates is provided. The kit comprises a polysulfated compound (such as, for example, dextran sulfate). In an embodiment, the kit further comprises a basal medium. In an embodiment, the kit further comprises factors known to support pluripotent stem cell growth and/or viability (such as, for example, FGF2, insulin, albumin, or glutamine).

In some embodiments, instructions for use of the kit to expand pluripotent stem cells in vitro are provided. The instructions may comprise one or more protocols for: media formulations; culture conditions, such as time, temperature, and/or gas incubation concentrations; harvesting protocols; and protocols for identifying pluripotent stem cells.

The kit may further include materials useful to conduct the present method including culture plates, welled plates, petri dishes and the like.

Non-limiting embodiments are described by reference to the following examples which are not to be construed as limiting.

### Example 1: Methods

In Example 1, the methods used in the subsequent Examples are described.

Human Pluripotent Stem Cell Culture and Induction to Naïve-Like State

H9 hESC were obtained from the WiCell Research Institute, HES2 hESC were obtained from G. Keller (McEwen Centre for Regenerative Medicine/University Health Network), and WIBR3 and C1.15 GFP lines were obtained from the Weizmann Institute (Rehovot, Israel)³⁵. The HES2 and H9 cells were cultured on Geltrex™ LDEV-Free Reduced Growth Factor Basement Membrane Matrix (Geltrex™, Thermo Fisher Scientific) coated plates. To coat the plates, they were incubated with a 1:50 dilution of Geltrex™ in Dulbecco's Modified Eagle's Medium (DMEM, Thermo Fisher Scientific) for 30 minutes at 37° C. or overnight at 4° C. The H9 and HES2 hESC were seeded on the Geltrex™-coated plates in Nutristem® hESC XF Culture Medium (NS, Biological Industries) supplemented with 1× Penicillin-Streptomycin (Thermo Fisher Scientific). Where noted, hESC were cultured on feeder layers of irradiated mouse embryonic fibroblasts in serum replacement medium (SR) comprised of DMEM/F12 (Thermo Fisher Scientific), 20% KnockOut™ Serum Replacement (Thermo Fisher Scientific), 1× GlutaMAX™ (Thermo Fisher Scientific), 1× Non-Essential Amino Acids (Thermo Fisher Scientific), 1× Penicillin-Streptomycin, and 10 ng/mL FGF2 (Peprotech). TrypLE™ Express (Thermo Fisher Scientific) was used to passage the hESC as single cells at a split ratio of 1:12 to 1:24 every 5 to 6 d. All cell line stocks tested negative for mycoplasma contamination.

To convert H9 and HES2 hESC to an alternative pluripotent state, primed hESC were passaged onto feeder layers of irradiated mouse embryonic fibroblasts in medium as described previously³⁵ with modifications as follows. In brief, base medium contained DMEM/F12, 2% KnockOut™ Serum Replacement, 1× GlutaMAX™, 1× Non-Essential Amino Acids, 1× Penicillin-Streptomycin, 2.5 mg/mL AlbuMAX™ 2 (Gibco®), 1× N2 Supplement (Gibco®), 0.625 μL/mL Insulin (Sigma), and 50 μg/mL Ascorbic acid (Sigma). This base medium was supplemented with 20 ng/mL human Leukemia Inhibitory Factor (LIF, Peprotech), 2 ng/mL TGFβ1 (R&D), 8 ng/mL FGF2, 0, 0.1, or 1 μM PD0325901 (Reagents Direct), 3 μM CHIR99021 (Reagents Direct), 5 μM SP600125 (Santa Cruz Biotechnology or Stem Cell Technologies), 2 μM BIRB796 (Cayman Chemical or Stem Cell Technologies), and 1 μM Gö6983 (Santa Cruz Biotechnology or Stem Cell Technologies). Base media was prepared in 500 mL batches and aliquoted to 15 mL centrifuge tubes (Sarstedt) filled with 15 mL basal media and stored at 40 C for up to 2 weeks. Cytokines, small molecules, insulin, and ascorbic acid were added fresh to a basal media aliquot each day. Medium that is not aliquoted as described and made fresh may not perform as described in suspension culture.

Bioreactor and Suspension Culture

Dynamic suspension cultures were carried out in 6 well plates (Costar) on an orbital shaker and bioreactor runs were performed using the Micro-24 bioreactor system (Pall Corporation). To prevent cell attachment, suspension culture plates were pre-coated with 5% Pluronic® F-68 (Thermo Fisher Scientific) for 30 minutes at 37° C. or overnight at 4° C. Single cell dissociation was performed as described above using TrypLE™ Express treatment for 5 minutes at room temperature for naïve hPSC and at 37° C. for control primed hPSC. hPSC were seeded at a density of 2×10⁵ cells/mL in either NS medium, SR medium, or the treatment formulation supplemented with 10 μM Y-27632 (Reagents Direct) under normoxic (i.e. 21% O₂) conditions.

Two days after seeding (unless otherwise noted), daily 50% medium exchanges were initiated. Plates were placed at a 45° angle to settle aggregates at the bottom edges of each well for 3 minutes. Half of the spent medium was then removed from the culture surface and replaced with fresh medium. At the end of the culture period, aggregates were harvested and dissociated using a 5 minute TrypLE™ treatment at 37° C. and the cells were counted on a hemocytometer using a Trypan Blue viability exclusion stain (Thermo Fisher Scientific).

Shear Sensitivity Assay

To assess shear sensitivity, cells in adherent cultures were dissociated and seeded at a density of 5×10⁵ cells/mL into Pluronic® F-68 coated-plates in the presence of Y-27632 as described above. Cultures were carried out for 8 hours at an agitation rate of 90 RPM. “No-shear” controls were prepared similarly but incubated at 37° C. in static conditions. After 8 hours, images of cells were taken.

RNA Preparation and Analysis

RNA extraction was performed using the RNeasy® Mini Kit (Qiagen). Reverse transcription was conducted according to manufacturer's instructions (SuperScript™ II kit, Invitrogen™) using 1 μg of total RNA for each sample. Gene expression analysis was performed on an Applied Biosystems™ 7900 HAT Real-time PCR machine using SYBR® Green PCR master mix (Roche, Sigma). The cDNA of cells at inoculation (day 0) or of adherent primed hPSC was used as a relative reference using the delta-delta Ct method and expression levels of genes of interest were normalized to GAPDH expression.

Differentiation Protocols

Differentiation to ectoderm was conducted as previously described⁴⁵: Briefly, hPSC were cultured in Pluronic® F-68 coated 6-well plates at a density of 0.5×10⁵ cells/mL in DMEM medium supplemented with 5% KnockOut™ Serum Replacement, 0.1 mM non-essential amino acids, 1 mM sodium pyruvate (Thermo Fisher Scientific), 1% Penicillin-Streptomycin and 0.1 mM β-mercaptoethanol (Sigma). At day 3, a 70% medium exchange was performed. At day 5, aggregates were transferred to Geltrex™ coated tissue culture treated 6-well plates and incubated for 5 days in media comprised of half DMEM-F12 and half Neurobasal™ medium (Thermo Fisher Scientific) supplemented with B27 without Retinoic Acid (Thermo Fisher Scientific) and N2 supplements (Thermo Fisher Scientific), 0.005% bovine serum albumin (Sigma), 1 mM sodium pyruvate before staining for β-III-TUBULIN expression (Cell signaling).

Mesoderm/cardiac differentiation was conducted as previously described⁴⁶ with select modifications. Briefly, hPSC aggregates were formed by seeding a single cell suspension of hPSC at 1.2×10⁶ cells per well into a Pluronic® F-68 coated 6 well-plate on an orbital shaker with 10 μM Y-27632 in NS medium (Biological Industries). After 2 days, a full medium exchange was performed to a cardiac differentiation base medium of StemPro® (Thermo Fisher Scientific) supplemented with 1× GlutaMAX™, 50 μg/mL Ascorbic acid, 1% Penicillin-Streptomycin, 150 μg/mL Transferrin (Sigma-Aldrich), and 0.04 μL/mL monothioglycerol (Sigma-Aldrich). Medium exchanges were performed at days 1, 4, 8, and 12, with different cytokine cocktails at each medium exchange that corresponded with specific stages of cardiac development. Cytokine cocktails were as follows: 5 ng/mL bFGF, 10 ng/mL BMP4 (R&D Systems), and 3 ng/mL Activin A (R&D Systems) at Day 1; 10 ng/mL VEGF (R&D Systems) and 2 μM IWP2 (Reagents Direct) at Day 4; and 10 ng/mL VEGF and 5 ng/mL bFGF at days 8 and 12. Cells were cultured under hypoxic conditions (5% O₂) from day 0 to 12 and under normoxic conditions from day 12-16. Aggregates were dissociated with TrypLE™ at day 16 and stained for cardiac TROPONIN-T (Thermo Fisher Scientific antibody MS295P).

Endoderm and pancreatic progenitor differentiation followed a variant of a published protocol^{39,40}. Briefly, pluripotent aggregates formed from hPSC seeded into suspension at 2×10⁶ cells/mL and cultured for 3 days in NS media (as described above, unless otherwise noted) were washed with phosphate buffered saline (Thermo Fisher Scientific) and cultured for 3 days in a basal medium of RPMI 1640 (Thermo Fisher Scientific), 1× GlutaMAX™, 1% Pen/Strep, 7.8×10⁻³ μL/mL monothioglycerol, and 3 μL/mL “SFD”. “SFD” contains 75% Iscove's Modified Dulbecco's Medium (Thermo Fisher Scientific) and 25% F12 (Thermo Fisher Scientific), supplemented with 1× N2 supplement, 1× B27 without Retinoic Acid, 25 μL/mL 20% Bovine Serum Albumin solution (Wisent). At day 0 of differentiation, 2 nM CHIR99021 and 100 ng/mL Activin A (produced in-house) were added to this medium. On day 1 and 2, 5 ng/mL bFGF, 100 ng/mL Activin A, and 50 μg/mL Ascorbic acid were added. Medium exchanges were performed daily. At day 3, cells were stained for C-KIT (BD Biosciences) and CXCR4 (BD Biosciences) protein expression and analyzed by flow cytometry. From day 3 to 12, the basal differentiation medium used consisted of DMEM, 1× GlutaMAX™, 1% Pen/Strep, 1% B27 without Retinoic Acid, 1× sodium pyruvate. On day 3 and 4, the base medium was supplemented with 50 ng/mL FGF10 (R&D Systems). On day 5 and 6, the base medium was supplemented with 0.25 μM KAAD-cyclopamine (Toronto Research Chemicals), 50 ng/mL FGF10, 2 μM Retinoic Acid (Sigma-Aldrich), 100 nM PDBu (Cedarlane), 200 nM LDN-193189 (Reagents Direct), and 50 ug/ml Ascorbic acid. Starting on day 7 the medium was supplemented with 50 ng/mL EGF, 10 mM nicotinamide (Sigma-Aldrich), 50 ng/mL NOGGIN, 50 ug/mL ascorbic acid. Medium was changed every second day. At day 12, cells were harvested and stained for NKX6.1 (DSHB) and PDX1 (R&D Systems) protein expression.

Cell Staining, Flow Cytometry, and Immunocytochemistry

For surface staining, dissociated cells were resuspended in Hank's Buffered Saline Solution (HBSS, Thermo Fisher Scientific) supplemented with 2% FBS (Gibco®) and incubated with CXCR4, C-KIT, and CD24 (BD Biosciences) antibodies at a 1:100 dilution for 30 minutes. Cells were then washed and resuspended in HBSS and 7AAD (Thermo Fisher Scientific) at a 1:1000 dilution.

For intracellular staining, dissociated cells were fixed with 4% paraformadehyde (Electron Microscopy Sciences) for 10 minutes and permeabilized with 0.1% Triton™ X-100 (Sigma-Aldrich). Cells were then stained with the primary antibody (OCT4, BD Biosciences; SOX2, R&D; FOXA2, Abnova; cTNT, Thermo Fisher Scientific; TUBB3, Cell Signaling; PDX1, R&D; NKX6.1, DSHB) for 25 minutes followed by the secondary antibody for 25 minutes at 4° C. Stained cells were analyzed on a FACSCanto™ II (BD) or FACS Fortessa™ (BD) flow cytometer.

For immunocytochemistry, samples were prepared as described for intracellular staining and imaged on an EVOS® microscope (Thermo Fisher Scientific).

Growth Rate Calculations

Growth rates were calculated by subtracting the initial cell density from the logarithm of the final density and dividing this value by the duration of the exponential growth phase.

Medium Utilization Rate Calculations

Medium samples (1 mL) were collected and frozen immediately prior to feeding suspension cultures. Samples were thawed and analyzed using a Bioanalyzer (Nova Biomedical). Apparent metabolic rates (qApp) were calculated at each time point (t) collected based on the concentration (c) of each metabolite one day after a half medium exchange and the average viable cell density (VCD) at that time point:

Viable cell density was estimated based on a 1 day lag phase post-seeding followed by exponential expansion to the cell density measured at day 6 of expansion. CO is the concentration of metabolite in fresh medium added at each half medium exchange. To account for the presence of GlutaMAX™, which degrades into glutamine in the presence of dipeptidases secreted by cells, CO of glutamine and glutamate was set as the maximum concentration of these metabolites when calculating metabolic rates. The limitation of this concentration and density averaging approach is that it linearly approximates exponential growth.

Oxygen consumption rate was measured using the MitoXpress® Xtra oxygen tracker kit (Luxcel Biosciences) according to manufacturer instructions.

Karyotype Analysis

hPSC were cultured in 5i medium for 5 passages and suspension 4i medium for 3 passages. Aggregates were dissociated and seeded into adherent conditions for karyotype analysis. G-Band karyotyping was performed by WiCell.

Statistical Analysis

Statistical analysis was performed using the JMP software (SAS). Parametric tests were used for qpcr, doubling time, and maximum density experiments, with the Student's T-test used for 2 treatment experiments and Tukey's HSD test used for experiments with 3 or more treatments. A non-parametric test (Kruskal-Wallis) was used for colony formation experiments since this assay was developed specifically for this study. Literature does not exist supporting an expected distribution of hPSC suspension colony formation efficiency, and a normal distribution was not assumed. * signifies p<0.05 unless otherwise noted. The linear regression model was developed in Excel (Microsoft®).

Pluripotent Stem Cell Culture with and without Dextran Sulfate

In brief, HES2 were cultured on Geltrex™ LDEV-Free Reduced Growth Factor Basement Membrane Matrix (Thermo Fisher Scientific, 1:50 dilution in DMEM) coated plates in Nutristem® hESC XF Culture Medium (Biological Industries, “NS”) or E8 Medium (Thermo Fisher Scientific), supplemented with 1× Penicillin-Streptomycin (Thermo Fisher Scientific). Plates were coated with Geltrex™ for 30 minutes at 37° C. Cells were passaged 1:24 every 6 days by dissociation with TrypLE™ Express (Thermo Fisher Scientific) at 37° C. for 4-5 minutes. All cell line stocks were confirmed negative for mycoplasma contamination. Conversion to a “naïve” hPSC state was performed as described previously³⁵. Dextran sulfate compounds (DS, Sigma) were prepared by dilution in deionized water at a stock concentration of 100 mg/mL followed by filter sterilization.

Suspension Expansion of PSC with and without Dextran Sulfate

Six well culture plates were pre-coated with 5% Pluronic F-68® (Thermo Fisher Scientific) for 30 minutes at 37° C. or overnight at 4° C. Dissociation of PSC to single cells was carried out by TrypLE™ Express (Thermo Fisher Scientific) treatment for 5 minutes at room temperature. Single cells were seeded into the Pluronic F-68® coated 6-well plates that were then placed on an orbital shaker with 10 μM Y-27632 (Reagents Direct) in normoxic (21% 02) conditions. Cells were seeded at densities of 2×10⁵ cells/mL unless otherwise noted. Medium was exchanged by angling the plate at a 45° angle and allowing aggregates to settle onto the bottom edge. Two days after seeding, half of the growth medium was removed and replaced each day. Cells were harvested at the end of culture by dissociation with a 5 minute TrypLE™ treatment at 37° C. and cell counts were performed by Trypan Blue exclusion (Thermo Fisher Scientific). Bioreactor experiments were conducted following manufacturer instructions for operating the Ambr™-24 bioreactor system (Sartorius). Cell dissociation and medium exchange were performed as described above. Bioreactors were operated at 450 rpm and seeded with 4×10⁵ cells/mL unless otherwise noted. Aggregates were analyzed using the Mastersizer™ 3000 particle analyzer (Malvern) according to manufacturer's instructions. Unless otherwise noted, the 560 μm aperture was used for analysis. Aggregates were dissociated and seeded into adherent conditions after 3 passages in suspension with DS. Directed differentiation into all three germ layers was performed as described previously for endoderm⁴⁰, mesoderm⁴¹, and ectoderm⁴² lineages. G-Band karyotyping was performed by WiCell.

Flow Cytometry and Immunocytochemistry of hPSCs with and without Dextran Sulfate

Apoptosis analysis was conducted using Annexin V staining. Dissociated cells were resuspended in Hank's Buffered Saline Solution (Thermo Fisher Scientific) supplemented with 2% fetal bovine serum (Gibco) (HF). Cells were washed once in HF and then resuspended in the Annexin V Binding Buffer (BD Biosciences) solution with the antibody (BD Biosciences, Annexin V-647, 1:100 dilution) for 15 minutes. Cells were washed in binding buffer again and resuspended in 7AAD (Thermo Fisher Scientific, 100 μg/mL) viability stain to distinguish necrotic cells from apoptotic cells. For intracellular staining, dissociated cells were fixed with 4% paraformadehyde (Electron Microscopy Sciences) for 10 minutes and permeabilized with 0.1% Triton™ X-100 (Sigma-Aldrich). Cells were then stained with the primary antibody for 25 minutes at 4° C. followed by the secondary antibody for 25 minutes at 4° C. Three wash steps with HF were included between each step. Cells were analyzed on a FACS Canto™ II (BD) or FACS Fortessa™ (BD) flow cytometer. Analysis was performed with FlowJo® (Tree Star). For immunocytochemistry, undissociated samples were prepared as described for intracellular staining and imaged on an EVOS™ microscope (Thermo Fisher Scientific).

Statistical Analysis of Data on hPSC Aggregates with and without Dextran Sulfate

Statistical analysis was performed in Excel software (Microsoft®). Student's t-test was used to compare treatments in FIG. 15E, 16C, 16D, 16E. Coefficient of variation was calculated by estimating the volume of each aggregate from its diameter, calculating the standard deviation of each treatment, and normalizing to the mean. 2-sided F-test was used to compare coefficients of variation. Error bars represent standard deviation. * denotes p<0.05 unless otherwise noted.

Culture with in 4i Medium with Dextran Sulfate

HES2 and H9 hPSC were cultured in adherent conditions in 5i medium for >5 passages. Cells were dissociated with TrypLE™ as described previously and seeded as single cells into 4i medium in the presence of ROCK inhibitor into orbital shaker suspension culture plates (pre-treated with 5% Pluronic® F-68) in the presence of dextran sulfate at seeding. Half of the medium volume was exchanged every day starting on the second day by tilting the plate at a 45 degree angle and allowing cells to settle before removing half of the medium and replenishing it with fresh 4i medium without dextran sulfate.

### Example 2: 5i-hPSC have Altered Colony Morphology, Transcription Factor Expression, and Surface Marker Expression

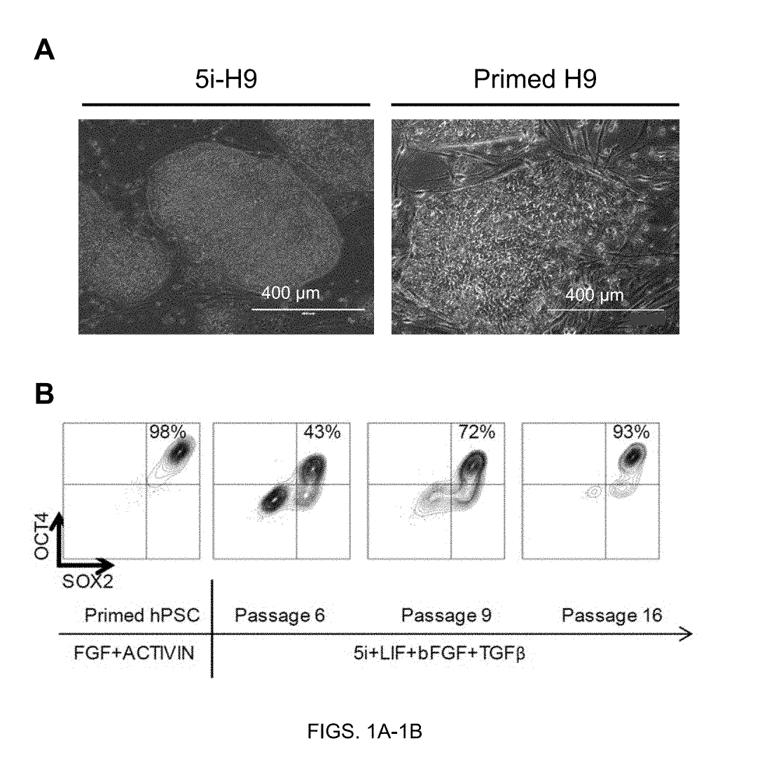

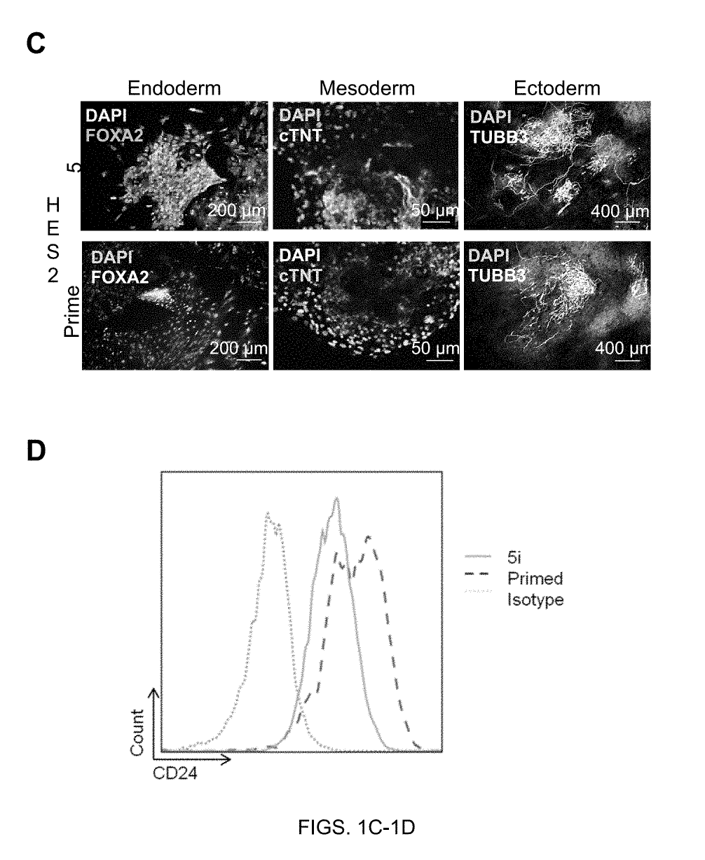

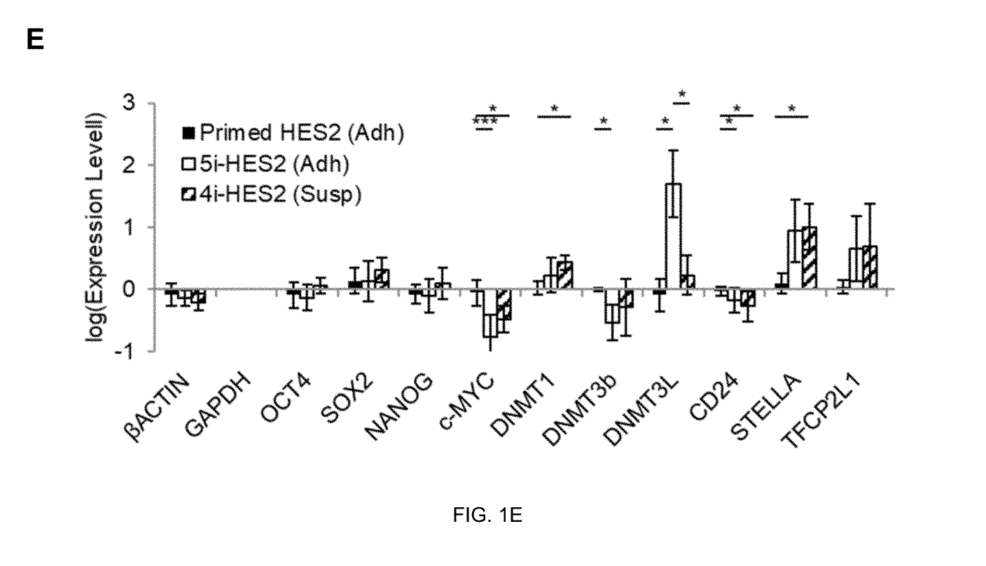

A protocol was tested that has been reported to maintain “naïve” hPSC with enhanced bioprocessing attributes in a cocktail containing 5 inhibitors (5i), including PKC inhibition and p38 inhibition via the BIRB796 molecule³⁵. After culturing H9 and HES2 hPSC in the 5i condition, the appearance of colonies with a raised morphology (FIG. 1A) were observed that maintained high levels of expression of key pluripotency factors OCT4 and SOX2 for >15 passages (FIG. 1B). It was observed that following the transfer of “primed” hPSC from conventional hPSC medium to the 5i condition, population expression of OCT4/SOX2 pluripotency markers initially decreased and then recovered and stably increased (FIG. 1B). These stable “5i-hPSC” demonstrated functional pluripotency by maintaining the capacity to differentiate into cell types of all three germ layers (FIG. 10). Consistent with previously published findings⁴³, reduced gene and protein expression of the surface marker CD24 in 5i-hPSC were observed compared to primed hPSC (FIG. 1 D-E). In line with published reports on “naïve” hPSC conversion^{25,30,35}, transcription factors DNMT1, DNMT3L, and STELLA were upregulated, while c-MYC and DNMT3B were downregulated in 5i-PSC. It was observed that changes in gene expression levels for core pluripotency factors OCT4, SOX2, and NANOG were negligible after transitioning primed hPSC to 5i-hPSC (FIG. 1E), demonstrating that these conditions maintain a core pluripotency gene regulatory network.

### Example 3: 5i-hPSC have Enhanced Bioprocessing Properties that Facilitate Increased Yields in Suspension Culture

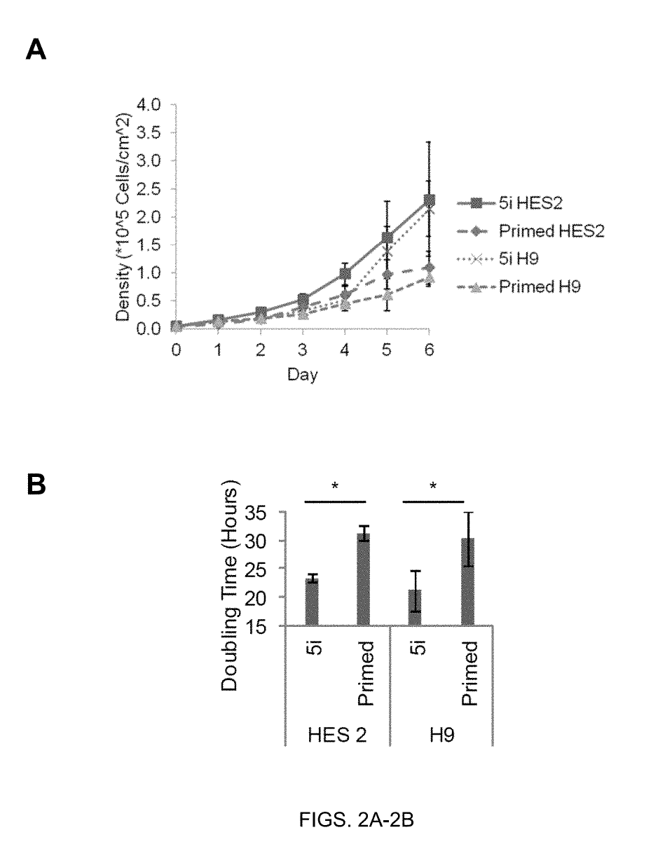

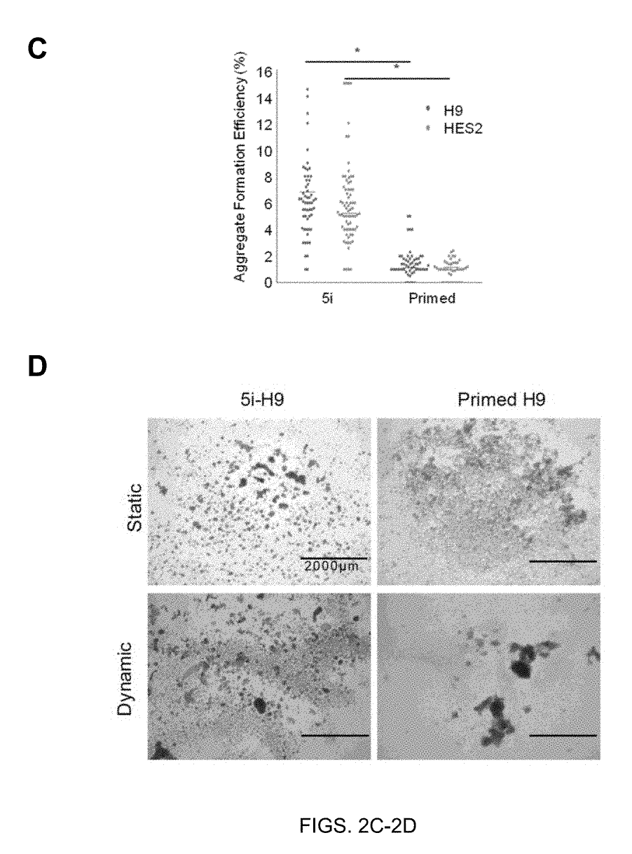

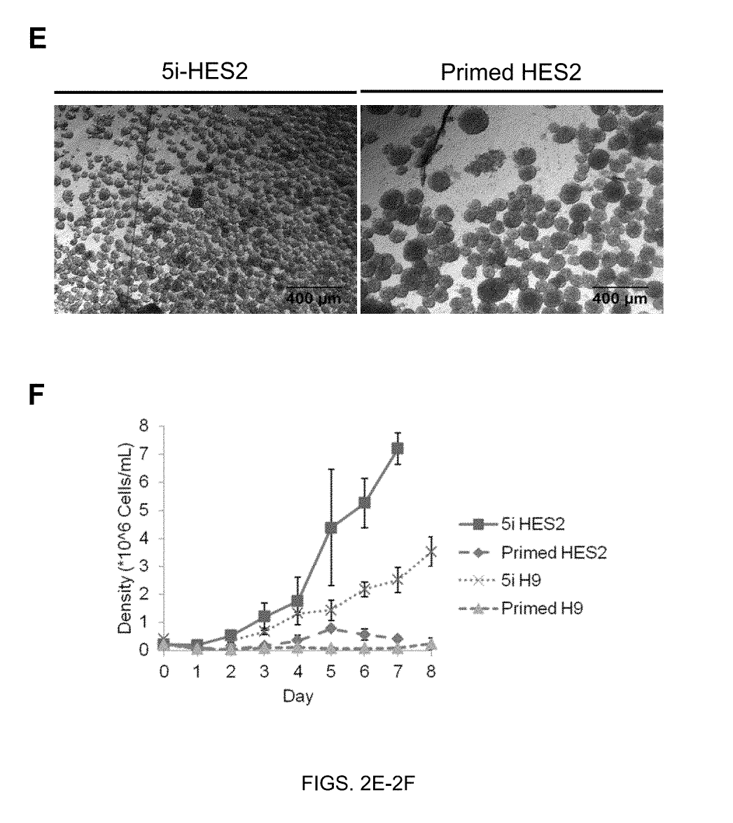

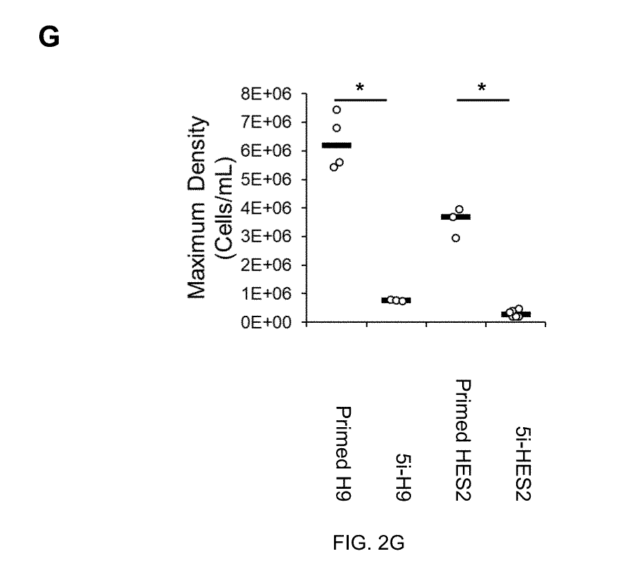

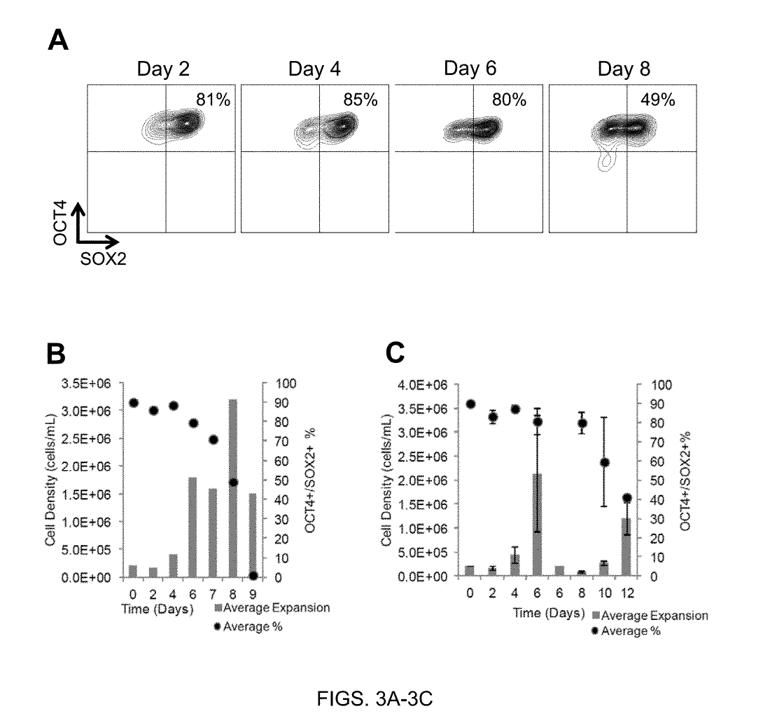

The suitability of 5i-hPSC for high yield culture in suspension bioreactors was evaluated by characterizing their growth kinetics, ability to form aggregates in static suspension, and agitated suspension survival and expansion. In preparation for suspension expansion, the growth rate of feeder-based adherent 5i-hPSC was calculated. 5i-hPSC exhibit higher proliferation rates than primed hPSC (FIG. 2A), leading to twice as many cells in 5i-hPSC cultures at day 6 post-seeding and significantly lower doubling times. 5i-hPSC were observed to form rounded 3-dimensional colonies when confluence was reached, and a plateau stationary phase of growth was not clearly observed. Both 5i-H9 and 5i-HES2 had significantly lower doubling times than untreated hPSC. 5i-H9 hPSC grew at average doubling times (exponential phase) of 21.1±3.6 hours in adherent conditions as compared to 30.3±4.7 hours for primed cells, whereas 5i-HES2 hPSC grew at average doubling times of 23.3±0.74 hours and primed cells doubled every 31.2±1.3 hours (FIG. 2B).

In static suspension conditions, aggregate formation characteristics of 5i-hPSC were compared to primed hPSC. Seeded as single cells at low density (100 cells/well in 96 well plate), suspension aggregate formation efficiencies were significantly higher in 5i-hPSC than in primed hPSC (4±1 and 6±1 fold higher using the HES2 and H9 cell lines respectively) (FIG. 2C). This transition to suspension culture results in a feeder-free culture system. It is expected that eliminating the feeder layer, in addition to transitioning to the 5i medium composition which contains almost completely defined components (contains purified Albumin) reduces variability between manufacturing runs.

To evaluate 5i-hPSC survival and expansion as aggregates under dynamic suspension conditions, primed and 5i-hPSC were plated as single cells in ultra-low attachment dishes that were then placed on orbital shakers or into a small-scale bioreactor. After 8 hours in orbital shaker suspension, 5i-H9 formed aggregates of varying sizes under both dynamic (orbital shaker condition) and static control conditions. Primed H9 formed small aggregates in the static control. However, in dynamic conditions larger, irregular, and dark, clumps formed (FIG. 2D), suggesting the presence of dead cells and debris. After 72 hours in suspension bioreactor culture, 5i-hPSC aggregate size visually appeared homogeneous compared to the heterogeneity observed in primed hPSC aggregate sizes (FIG. 2E). A quantitative analysis of aggregate diameters confirmed this visual observation, determining that at 72 hours 5i-hPSC aggregates displayed an average diameter of 51±18 μm while primed hPSC displayed an average aggregate diameter of 113±43 μm (FIG. 7). These results also demonstrated that 5i-hPSC form smaller initial aggregates than primed aggregates. These findings indicate a greater capacity for suspension growth from aggregates formed from 5i-hPSC, given that there is typically a maximum aggregate size above which cell proliferation is no longer supported⁴⁴.

The dynamic suspension studies described above were performed using culture plates on orbital shakers and in the micro-bioreactor system. The growth curves for primed hPSC and 5i-hPSC had varied lag phase timing, growth rate, and maximum cell density (FIG. 2F). Cell densities peaked at days 5-6 for primed HES2, which was used as the baseline for further comparisons. 5i-HES2 and H9 density peaked at days 7 and 8 (FIG. 2G). Specifically, the peak 5i-HES2 density (6.32±0.9×10⁶ cells/mL) represented a 25-fold expansion and was nearly 6 fold greater than that of primed HES2 at their peak density (7.73±0.3×10⁵ cells/mL). Similar growth curves were obtained for the 5i-H9 cell line, which reached a maximum cell density of 3.5±0.5×10⁶ cells/mL. Dramatically higher proliferation rates were observed in 5i-hPSC, as represented by their average exponential growth doubling times in suspension: 29.0±1.3 and 34.9±1.9 hours for 5i-HES2 and 5i-H9 respectively, and 49.2±1.2 hours for primed HES2

### Example 4: Culture Optimization Enables Long-Term, High-Yield Maintenance of the Pluripotent State in Suspension

While high cell expansion in short term 5i-hPSC bioreactor cultures was achieved, a gradual loss of the pluripotent phenotype (OCT4+/SOX2+) was observed that dropped below 50% by day 8. (FIG. 3A). This observation is in contrast to what has been reported during suspension expansion of primed hPSC¹⁸. To address this loss of pluripotency and enable high-yield, long-term hPSC expansion in suspension, various culture parameters were examined to identify whether loss of pluripotency was due to endogenous interactions, missing feeder layer interactions, or one of the components in 5i medium.

In short-term bioreactor studies, high levels of pluripotent cells were observed at the peak cell densities reached in primed hPSC cultures, but not at the peak densities achieved in 5i-hPSC cultures (FIG. 3B). To develop a bioreactor protocol to maintain pluripotency of 5i-hPSCs, a strategy was explored in which cells were passaged at the last day on which >80% of cells expressed a pluripotent phenotype (day 6), with the hypothesis that beyond this time point, large aggregate size inhibits pluripotency through media limitations in 5i conditions. However, a pluripotent phenotype was unable to be maintained in 5i-hPSC cultured in suspension beyond 2 passages with this strategy (FIG. 3C). It was next investigated whether optimization of dissociation timing may improve pluripotency by decreasing maximum aggregate size or increasing nutrient level per cell in culture, as has been recently suggested⁴⁵. On days 4, 5, 6, and 7 of 5i-hPSC suspension, the aggregates were either passaged to reduce aggregate size or diluted 1:10 in fresh medium to reduce the bulk cell density (see schematic in FIG. 8A). However, after 4-7 days in both the dissociation experiment and the dilution experiment, no condition was able to maintain the pluripotent phenotype (FIG. 8B). Given that the medium being used in the bioreactor maintains 5i-hPSC pluripotency in adherent feeder-based conditions, it was speculated that the loss of the pluripotent phenotype was due to either the transition from adherent to suspension culture or to the loss of a specific interaction with the feeder layer.

To test this hypothesis, a 96 well plate platform was designed to screen the effects of medium components on 5i-hPSC pluripotency in suspension cultures and in feeder-free adherent cultures (FIG. 8A). To examine the effects of adherent culture on pluripotency, a Geltrex™ coating was used to obtain an adherent culture condition in the absence of a feeder layer. On Geltrex™, 5i-hPSC were still unable to propagate while maintaining a pluripotent phenotype (FIG. 3D, adherent “1PD” condition) and rapid acquisition of a differentiated phenotype was observed, suggesting that adhesion alone does not maintain pluripotency in the 5i medium. Next, it was confirmed that suspension (low-adhesion coating) culture in the 96 well plate accurately replicated the kinetics of pluripotency loss (FIG. 8B) that was previously observed in the bioreactor, thereby providing a reliable platform to screen for the criticality of medium components for maintenance of the pluripotent phenotype in suspension. A screen was performed evaluating each of the components in 5i medium by either removing the component in question, reducing its concentration by half, or doubling its concentration. The most striking finding from this screen was that feeder-free adherent maintenance of pluripotency improved upon removal of the ERK1/2 inhibitor (PD) from the 5i medium formulation (FIG. 9). This finding was validated by performing a dose response experiment to determine the effect of PD concentration on the expression of the pluripotent phenotype in both feeder-free adherent and suspension conditions (FIG. 3D). A linear regression was performed to identify significant interaction effects between PD level and the growth format (Table 1).

TABLE 1
Model of PD effect: A linear regression model indicates the significance of PD level on
OCT4/SOX2 expression. Growth format and interaction effects were also significant.
Regression Statistics

| | |
|---|---|
| Multiple R | 0.707981 |
| R Square | 0.501238 |
| Adjusted R Square | 0.472463 |
| Standard Error | 18.48842 |
| Observations | 56 |

ANOVA

| | df | SS | MS | F | Significance F |
|---|---|---|---|---|---|
| Regression | 3 | 17862.93 | 5954.31 | 17.41935 | 5.88E−08 |
| Residual | 52 | 17774.72 | 341.8216 | | |
| Total | 55 | 35637.65 | | | |

| | | Standard | | | | |
|---|---|---|---|---|---|---|
| | Coefficients | Error | t Stat | P-value | Lower 95% | Upper 95% |
| Intercept | 51.6144 | 3.507931 | 14.71363 | 3.55E−20 | 44.57522 | 58.65358 |
| PD | −27.0584 | 4.375158 | −6.18455 | 9.83E−08 | −35.8378 | −18.279 |
| Growth | −14.6832 | 4.960963 | −2.95975 | 0.004628 | −24.6381 | −4.7283 |
| Format | | | | | | |
| PD * Format | 18.8952 | 6.187408 | 3.053815 | 0.003558 | 6.479257 | 31.31114 |

It was determined that PD level was negatively correlated to pluripotent phenotype, and interaction effects were observed with growth format. A number of molecules, recently reported to further stabilize the “naïve” pluripotent state, were tested to determine whether hPSC maintenance was possible under feeder-free conditions in the presence of PD. The CGP small molecule was tested to inhibit the SRC pathway which has been shown to lead to the raised colony morphology characteristic of “naïve” hPSC³⁰. LPA, a small molecule shown to activate the YAP/TAZ pathway and enable “naïve” hPSC morphology³⁴ was also tested. However, neither of these molecules supported the maintenance of the pluripotent phenotype in the presence of PD (FIG. 10A). Interestingly, PD inhibits pluripotency in suspension, yet primed hPSC converted to an alternative state in the absence of PD (using the 4 inhibitor formulation) did not have enhanced suspension expansion properties. In suspension, they show poor aggregation with a dark, heterogeneous aggregate morphology typical of primed hPSC (FIG. 10B). These findings demonstrate the critical role that the ERK pathway plays in this cell state conversion as well as maintenance of pluripotency in suspension.

Next, using hPSC initially cultured in adherent conditions in the 5i formulation, expansion of these hPSC was tested in orbital shaker suspension culture in the 4i medium. “4i-hPSC” were cultured in suspension for 5 passages, achieving an average cell density of 5.2±1.1×10⁶ cells/mL at passage 5 while maintaining a 67±9% pluripotent phenotype (FIG. 3E). These results represent a 25±5.5-fold cell expansion in 6 days that is 5.7±0.2 fold greater than that achieved using primed HES2 in the same time period. hPSC cultured in 5i adherent culture for 5 passages and 4i suspension culture for 3 passages do not display karyotypic abnormalities (FIG. 11A) and are able to differentiate into cells of all 3 germ layers (FIG. 11B). To test the robustness of the 4i-hPSC system, an expansion was carried out using the H9 cell line, which was unable to expand in NutriStem™ (NS) medium and expands only moderately in conventional serum-replacement HES medium (approximately 1.5 fold expansion after one passage) (FIG. 12A). In suspension, 4i-H9 expanded nearly 9 fold in 6 days while maintaining nearly 80% pluripotent marker expression (FIG. 12B). These results show that the use of the 4i-hPSC culture system enables suspension bioprocessing of hPSC lines that are not readily maintained in the suspension. Suspension expansion of two additional lines, WIBR3 hESC and C1.15 hiPSC (FIG. 12C) in 4i conditions led to significantly enhanced fold expansion (19.0±2.0 and 21.5±2.5 fold expansion respectively) relative to primed hPSC, demonstrating that this technique is applicable to other hPSC lines including iPSC.

### Example 5: Distinct Adhesion Molecule Expression and Medium Utilization in Suspension in 4i-hPSC

In suspension, altered state hPSC had improved single cell survival, more efficient aggregate formation, and enhanced shear tolerance compared to primed hPSC, all of which could be related to adhesion signaling. Given that a number of reports have linked cell adhesion signaling to pluripotency^{27,46,47}, it was hypothesized that the enhanced suspension properties of 4i-hPSC may be due to reduced expression of adhesion molecules, which in turn lower the cells' dependence on adhesion for survival and maintenance of pluripotency. To identify whether adhesion related genes are differentially expressed between 4i-hPSC and primed hPSC, a panel of adhesion molecules were analyzed by qPCR. In the genes that displayed differential expression, most had significantly different expression levels in the 4i-hPSC in suspension condition than in both adherent primed and adherent 4i-hPSC (p<0.1, Tukey's HSD test). Notably, several genes related to the extracellular matrix as well as a variety of integrin molecules (ITGA7, ITGA8, ECM1, VTN, TGFBI, and HAS1) were observed to have differential expression between 4i-hPSC and primed cells in adherent conditions (p>0.1, Tukey's HSD test) (FIG. 4A). Differentially expressed molecules were validated by flow cytometry and it was found that ICAM1, ITGA5, and ECM1 are differentially expressed between suspension 4i-hPSC and primed hPSC (FIG. 13). These markers could supplement recent efforts to characterize this altered pluripotent state.

Next, the metabolic demand and activity of 4i-hPSC were compared to primed hPSC. During a 6-day suspension culture of 4i-HES2 and primed HES2, medium was sampled every day and analyzed for select metabolite levels. Since metabolic usage in hPSC is dependent on available metabolites⁴⁸ primed HES2 were cultured both in NS medium optimized for feeder free growth as well as Serum Replacement (SR) medium whose base is similar to 4i and contains similar metabolite levels. Apparent metabolic rates (qApp) were calculated at each sampling point (FIG. 4B). A number of trends were observed between 4i-HES2 and primed HES2, as well as between the two primed conditions tested (NS and SR). Glucose levels dropped from their initial level in all conditions reaching a steady level by day 2 in primed conditions but not in 4i-hPSC. While 4i-hPSC continued to grow to higher densities even at lower glucose levels, the steady glucose levels in primed hPSC suggested that primed cells may require higher glucose levels for growth. Indeed, glucose uptake per cell was much lower for 4i-hPSC than primed cells, though NS medium had higher specific uptake than SR medium, suggesting that nutrient availability may contribute to these trends as well. Glutamine and glutamate levels appeared relatively unchanged in the medium of primed conditions. In 4i medium, glutamine and glutamate dropped after day 3 at the same rate as cell expansion (FIG. 4B). Interesting trends were observed in lactate and ammonium levels, common waste products of cell culture. Lactate production was higher in primed cultures grown in NS medium than either primed SR medium or 4i-hPSC medium, and its concentration rose above 15 mM at day 5. In contrast, ammonium levels were highest in 4i-hPSC cultures, reaching 2.0 mM at day 6, while primed culture levels appeared to stabilize near 0.5 mM from day 2 onwards (FIG. 4B). Furthermore, adherent primed hPSC were observed to have a reduced oxygen consumption rate relative to adherent 5i-hPSC (FIG. 14). These altered state hPSC therefore display a metabolic usage distinct from that of primed hPSC that is independent from base nutrient differences between the conditions.

### Example 6: Directed Differentiation of 4i-hPSC to Pancreatic Progenitors does not Require Repriming

The capacity for alternative state hPSC to differentiate to pancreatic progenitors, a cell type of significant clinical interest³⁹ was tested. Directed differentiation of pancreatic progenitors from hPSC is achieved by the staged addition of molecules that are intended to mimic development³⁹ (FIG. 5A). Initial attempts to differentiate adherent 5i-hPSC (FIG. 5B) and recent reports on differentiation of “naïve” hPSC^{49,50} suggested that current differentiation protocols for primed hPSC result in low purity or cell yield when applied to 5i-hPSC. It was hypothesized that 5i-hPSC could be differentiated to clinically relevant lineages by culturing 5i-hPSC in primed hPSC conditions prior to differentiation. Indeed, it was found that “repriming” adherent 5i-hPSC 1 day prior to differentiation increased the fraction of definitive endoderm cells at day 3 of differentiation (FIG. 5B). These results verify that 5i-hPSC can be re-primed to differentiate in a similar manner as conventional primed hPSC.

Next, conditions were sought that would enable suspension differentiation. While a two-day repriming strategy enabled suspension differentiation of 5i-hPSC, it was found that the 4i-hPSC formulation could be efficiently differentiated towards pancreatic progenitors without a repriming step. Both 2 day repriming with NS feeder-free medium⁵¹ as well as 4i-hPSC differentiation towards definitive endoderm (C-KIT+/CXCR4+) resulted in high purity (>90%) definitive endoderm phenotype after 3 days (FIG. 5C-5D) with the capacity to generate Pdx1+Nkx6.1+ endocrine pancreatic cells after 12 days (FIG. 5E). It was observed that by day 12, re-primed 5i-hPSC and 4i-hPSC were able to form PDX1/NKX6.1 double positive cells, albeit at different yields and purities (FIG. 5E). Comparing outcomes from these strategies did not result in statistically significant differences in purity, expansion, or yield after 3 days of definitive endoderm differentiation, though 4i-hPSC had a significantly greater number of cells after 12 days of differentiation relative to reprimed 5i-hPSC (p<0.01, Tukey's HSD), with no significant difference in purity. 4i-hPSC are thus capable of pancreatic progenitor differentiation.

### Example 7: Comparison of Novel Media and Method to Previous Outcomes

Not surprisingly, analyzing published reports of suspension expansion of mouse and hPSC (FIG. 6) illustrates that 4i-hPSC are distinct from traditional hPSC cultures in suspension expansion yield, intensification (i.e. cell density), and growth rates. The outcomes using the 4i-hPSC bioprocess far exceed those observed in any hPSC suspension expansion system to date, and are within the ranges seen in high yield mouse PSC systems (FIG. 6), as described in reference 52.

### Example 8: Chemically Controlled Aggregation of Pluripotent Stem Cells in Small-Scale Culture Systems

To guide stirred tank bioreactor studies, the concentration of dextran sulfate (DS) was first varied in small scale orbital shaker suspension culture using three molecular weight DS: 4 kDA (D4), 15 kDA (D15), and 40 kDA (D40). In each of these conditions, changes in PSC aggregation properties were observed compared to the PSC medium control (FIG. 15B). The addition of DS to the medium resulted in the formation of aggregates with significantly reduced diameters in a dose-dependent fashion (FIG. 15C, FIG. 17B). In addition, more homogeneous aggregate sizes were observed in the presence of DS, as evidenced by reduced standard deviations and lower coefficients of variation of aggregate volume (FIG. 17A). However, in the absence of DS, large, heterogeneous aggregates formed, with a small number of very large aggregates (diameter 150-700 μm) that contained a large fraction of the cells (FIG. 15C, FIG. 17B, FIG. 17C).

To maintain homogeneous aggregate sizes throughout the culture period, DS treatment is only required during PSC seeding. Similar aggregate sizes were observed at each time point in both conditions where PSC were treated with D40 either daily or only at cell seeding (FIG. 15D). PSC were seeded into static suspension conditions at low densities typically used for clonal expansion. In the presence of 100 μg/mL of all DS compounds, enhanced cell survival and aggregate formation were observed over untreated conditions (FIG. 15E).

### Example 9: Chemically Controlled Aggregation of Pluripotent Stem Cells in Scalable Culture Systems

The capacity for small, homogeneous DS aggregates to expand in culture was examined over a six day culture period. In suspension culture on an orbital shaker, small aggregates formed in D40 remained small and homogeneous throughout the six day expansion relative to the large and heterogeneous untreated aggregates (FIG. 16A). Translating these orbital shaker findings to scalable stirred suspension bioreactors for PSC culture confirmed that D40 at seeding was associated with reduced aggregate size throughout the process relative to untreated PSC (FIG. 16B). After 6 days in stirred suspension, untreated PSC displayed large, heterogeneous aggregate morphologies in both bioreactor geometries. Furthermore, in stirred tank bioreactors seeded with DS, PSC were successfully expanded for 3 passages while maintaining high expression of pluripotency markers Oct4 and Sox2 (>85%) (FIG. 17D). Importantly, after 9 passages (5 passages in adherent conditions, 4 passages in suspension), DS treated PSC retained a normal karyotype (FIG. 17E) and maintained the ability to differentiate to cell types from all three germ layers (FIG. 17F). These findings indicate that DS treatment enables uniform aggregate formation without causing a loss of pluripotency or introducing karyotypic instability, as described in reference 53.

The fate of large PSC aggregates that arise in control cultures was investigated to understand the benefit that DS conveys to PSC suspension expansion. By extending the suspension culture period to 10 days, untreated PSC cultures were observed to contain primarily large heterogeneous aggregates, while D40-treated conditions contain homogeneous small aggregates (FIG. 17G). The untreated, large, dark aggregates exhibited elevated expression of early apoptotic marker Annexin V (FIG. 16C). When dissociated and reseeded in suspension, D40 treated cells again formed homogenously small aggregates whereas control cells formed larger heterogeneous aggregates. The cell recovery following dissociation after 2 days in culture was significantly enhanced in the D40-treated PSC (FIG. 16D). Heterogeneous aggregation in the first passage preceded reduced cell recovery, lower pluripotency marker expression, and ultimately, lower cell yield after 6 days of a second passage (FIG. 16E). It appears that DS enhances process robustness by mitigating the risk that variable and difficult to control input factors cause large aggregate formation. These large aggregates would otherwise lead to reduced cell viability and expansion potential, and may impact downstream differentiation capacity. In addition to the HES2 line, DS is broadly applicable for aggregation control across different cell lines and culture conditions. The H9 hPSC line was cultured in a medium containing DS in both conventional PSC culture conditions as well as conditions supporting a “naïve” pluripotent state and it was found the DS is able to control H9 hPSC aggregation in both pluripotent states (FIG. 17H).

Similar to the effects of DS, it was observed that heparin and pentosan polysulfate also displayed aggregate control properties in PSC (data not shown). It was observed that while 1000 μg/mL D15 and D40 lead to the largest number of small aggregates, the total cell number is reduced in these conditions (FIG. 17I). This finding suggests that in addition to anti-apoptotic effects of DS, trade-offs should be considered between the increased growth potential of small hPSC aggregates, inhibitory maximum DS concentrations, the availability of pro-survival signals received by cells in small aggregates, and additional mechanisms of action.

DS could also enable strategies for the scalable translation of differentiation protocols requiring precise control of aggregate size. Encouragingly, DS requires no pre-treatment or adaptation for efficacy. DS consistently leads to homogeneous aggregation across different bioreactor geometries, suggesting that it may mitigate geometry-associated process translation challenges caused by different mixing, shear, and initial aggregation properties. Further optimization of this system may enable single cell suspension culture of PSC (FIG. 15A iv), which would resolve many of the issues around growth of adhesion dependent cells in suspension bioreactors.

### Example 10: Control of hPSC Aggregate Size with 4i Media and Dextran Sulfate

The 4i-hPSC culture system was further supplemented with dextran sulfate. Mean aggregate size and variance in aggregate size were both observed to decrease in suspension conditions treated with dextran sulfate relative to untreated conditions in both HES2 and H9 cell lines (FIG. 18).

Although the disclosure has been described with reference to certain specific embodiments, various modifications thereof will be apparent to those skilled in the art. Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. The scope of the claims appended hereto should not be limited by the preferred embodiments set forth in the above description, but should be given the broadest interpretation consistent with the present specification as a whole. The disclosures of all prior art recited herein are incorporated herein by reference in their entirety.

### DOCUMENTS CITED

1. Evans, M. J. & Kaufman, M. H. Establishment in culture of pluripotential cells from mouse embryos. Nature 292, 154-156 (1981).
2. Thomson, J. A. et al. Embryonic stem cell lines derived from human blastocysts. Science 282, 1145-1147 (1998).
3. Kirouac, D. C. & Zandstra, P. W. The systematic production of cells for cell therapies. Cell Stem Cell 3, 369-381 (2008).
4. Lipsitz, Y. Y., Timmins, N. E. & Zandstra, P. W. Quality cell therapy manufacturing by design. Nat. Biotechnol. 34, 393-400 (2016).
5. Baptista, R. P., Fluri, D. A. & Zandstra, P. W. High density continuous production of murine pluripotent cells in an acoustic perfused bioreactor at different oxygen concentrations. Biotechnol. Bioeng. 110, 648-655 (2013).
6. Fernandes-Platzgummer, A., Diogo, M. M., Baptista, R. P., da Silva, C. L. & Cabral, J. M. S. Scale-up of mouse embryonic stem cell expansion in stirred bioreactors. Biotechnol. Prog. 27, 1421-1432 (2011).
7. Cormier, J. T., zur Nieden, N. I., Rancourt, D. E. & Kallos, M. S. Expansion of undifferentiated murine embryonic stem cells as aggregates in suspension culture bioreactors. Tissue Eng. 12, 3233-3245 (2006).
8. Shafa, M. et al. Expansion and long-term maintenance of induced pluripotent stem cells in stirred suspension bioreactors. J. Tissue Eng. Regen. Med. 6, 462-472 (2012).
9. Kehoe, D. E., Lock, L. T., Parikh, A. & Tzanakakis, E. S. Propagation of embryonic stem cells in stirred suspension without serum. Biotechnol. Prog. 24, 1342-1352 (2008).
10. zur Nieden, N. I., Cormier, J. T., Rancourt, D. E. & Kallos, M. S. Embryonic stem cells remain highly pluripotent following long term expansion as aggregates in suspension bioreactors. J. Biotechnol. 129, 421-432 (2007).
11. Fok, E. Y. L. & Zandstra, P. W. Shear-Controlled Single-Step Mouse Embryonic Stem Cell Expansion and Embryoid Body-Based Differentiation. Stem Cells 23, 1333-1342 (2005).
12. Abranches, E., Bekman, E., Henrique, D. & Cabral, J. M. S. Expansion of mouse embryonic stem cells on microcarriers. Biotechnol. Bioeng. 96, 1211-1221 (2007).
13. Fernandes, A. M. et al. Mouse embryonic stem cell expansion in a microcarrier-based stirred culture system. J. Biotechnol. 132, 227-236 (2007).
14. Amit, M. et al. Dynamic suspension culture for scalable expansion of undifferentiated human pluripotent stem cells. Nat. Protoc. 6, 572-579 (2011).
15. Chen, V. C. et al. Scalable GMP compliant suspension culture system for human ES cells. Stem Cell Res. 8, 388-402 (2012).
16. Zweigerdt, R., Olmer, R., Singh, H., Haverich, A. & Martin, U. Scalable expansion of human pluripotent stem cells in suspension culture. Nat. Protoc. 6, 689-700 (2011).
17. Olmer, R. et al. Suspension culture of human pluripotent stem cells in controlled, stirred bioreactors. Tissue Eng. Part C. Methods 18, 772-784 (2012).
18. Lipsitz, Y. Y. & Zandstra, P. W. Human pluripotent stem cell process parameter optimization in a small scale suspension bioreactor. BMC Proc. 9, 010 (2015).
19. Kehoe, D. E., Jing, D., Lock, L. T. & Tzanakakis, E. S. Scalable stirred-suspension bioreactor culture of human pluripotent stem cells. Tissue Eng. Part A 16, 405-421 (2010).
20. Rossant, J. & Tam, P. P. L. New Insights into Early Human Development: Lessons for Stem Cell Derivation and Differentiation. Cell Stem Cell 20, 18-28 (2017).
21. Brons, I. G. M. et al. Derivation of pluripotent epiblast stem cells from mammalian embryos. Nature 448, 191-195 (2007).
22. Tesar, P. J. et al. New cell lines from mouse epiblast share defining features with human embryonic stem cells. Nature 448, 196-199 (2007).
23. Hanna, J. et al. Human embryonic stem cells with biological and epigenetic characteristics similar to those of mouse ESCs. Proc. Natl. Acad. Sci. U.S.A 107, 9222-9227 (2010).
24. Buecker, C. et al. A murine ESC-like state facilitates transgenesis and homologous recombination in human pluripotent stem cells. Cell Stem Cell 6, 535-546 (2010).
25. Takashima, Y. et al. Resetting transcription factor control circuitry toward ground-state pluripotency in human. Cell 158, 1254-1269 (2014).
26. Wang, W. et al. Rapid and efficient reprogramming of somatic cells to induced pluripotent stem cells by retinoic acid receptor gamma and liver receptor homolog 1. Proc. Natl. Acad. Sci. U.S.A 108, 18283-18288 (2011).
27. Xu, Y. et al. Revealing a core signaling regulatory mechanism for pluripotent stem cell survival and self-renewal by small molecules. Proc. Natl. Acad. Sci. U.S.A 107, 8129-8134 (2010).
28. Zimmerlin, L. et al. Tankyrase inhibition promotes a stable human naive pluripotent state with improved functionality. Development 143, 4368-4380 (2016).
29. Ware, C. B. et al. Derivation of naive human embryonic stem cells. Proc. Natl. Acad. Sci. U.S.A 111, 4484-4489 (2014).
30. Theunissen, T. W. et al. Systematic Identification of Culture Conditions for Induction and Maintenance of Naive Human Pluripotency. Cell stem cell 15, 524-526 (2014).
31. Ying, Q. L., Nichols, J., Chambers, I. & Smith, A. BMP induction of Id proteins suppresses differentiation and sustains embryonic stem cell self-renewal in collaboration with STAT3. Cell 115, 281-292 (2003).
32. Nichols, J., Silva, J., Roode, M. & Smith, A. Suppression of Erk signalling promotes ground state pluripotency in the mouse embryo. Development 136, 3215-3222 (2009).
33. Roode, M. et al. Human hypoblast formation is not dependent on FGF signalling. Dev. Biol. 361, 358-363 (2012).
34. Qin, H. et al. YAP Induces Human Naive Pluripotency. Cell Rep. 14, 2301-2312 (2016).
35. Gafni, O. et al. Derivation of novel human ground state naive pluripotent stem cells. Nature 504, 282-286 (2013).
36. Li, L. et al. Heparin promotes suspension adaptation process of CHO-TS28 cells by eliminating cell aggregation. Mol. Biotechnol. 47, 9-17 (2011).
37. Zanghi, J. A., Renner, W. A., Bailey, J. E. & Fussenegger, M. The growth factor inhibitor suramin reduces apoptosis and cell aggregation in protein-free CHO cell batch cultures. Biotechnol. Prog. 16, 319-325 (2000).
38. Hyoung Park, J., Sin Lim, M., Rang Woo, J., Won Kim, J. & Min Lee, G. The molecular weight and concentration of dextran sulfate affect cell growth and antibody production in CHO cell cultures. Biotechnol. Prog. 32, 1113-1122 (2016).
39. Nostro, M. C. et al. Efficient generation of NKX6-1+ pancreatic progenitors from multiple human pluripotent stem cell lines. Stem cell reports 4, 591-604 (2015).
40. Ungrin, M. D. et al. Rational bioprocess design for human pluripotent stem cell expansion and endoderm differentiation based on cellular dynamics. Biotechnology and bioengineering 109, 853-866 (2012).
41. Bauwens, C. L. et al. Geometric control of cardiomyogenic induction in human pluripotent stem cells. Tissue Eng. Part A 17, 1901-1909 (2011).
42. Fluri, D. A. et al. Derivation, expansion and differentiation of induced pluripotent stem cells in continuous suspension cultures. Nat. Methods 9, 509-516 (2012).
43. Shakiba, N. et al. CD24 tracks divergent pluripotent states in mouse and human cells. Nat. Commun. 6, 7329 (2015).
44. Wu, J., Rostami, M. R., Cadavid Olaya, D. P. & Tzanakakis, E. S. Oxygen transport and stem cell aggregation in stirred-suspension bioreactor cultures. PLoS One 9, e102486 (2014).
45. Kropp, C. et al. Impact of Feeding Strategies on the Scalable Expansion of Human Pluripotent Stem Cells in Single-Use Stirred Tank Bioreactors. Stem Cells Transl. Med. 5, 1289-1301 (2016).
46. Konze, S. A. et al. Cleavage of E-cadherin and beta-catenin by calpain affects Wnt signaling and spheroid formation in suspension cultures of human pluripotent stem cells. Mol. Cell. Proteomics 13, 990-1007 (2014).
47. Pieters, T. & van Roy, F. Role of cell-cell adhesion complexes in embryonic stem cell biology. J. Cell Sci. 127, 2603-2613 (2014).
48. Zhang, H. et al. Distinct Metabolic States Can Support Self-Renewal and Lipogenesis in Human Pluripotent Stem Cells under Different Culture Conditions. Cell Rep. 16, 1536-1547 (2016).
49. Geula, S. et al. Stem cells. m6A mRNA methylation facilitates resolution of naive pluripotency toward differentiation. Science 347, 1002-1006 (2015).
50. Irie, N. et al. SOX17 is a critical specifier of human primordial germ cell fate. Cell 160, 253-268 (2015).
51. Rodin, S. et al. Clonal culturing of human embryonic stem cells on laminin-521/E-cadherin matrix in defined and xeno-free environment. Nat. Commun. 5, 3195 (2014).
52. Lipsitz, Y. Y. et al. Modulating cell state to enhance suspension expansion of human pluripotent stem cells. Proc. Natl. Acad. Sci. U.S.A 115, 6369-6374 (2018).
53. Lipsitz, Y. Y. et al. Chemically controlled aggregation of pluripotent stem cells. Biotech. Bioeng. 119, 2061-2066 (2018).

We claim:

1. A method for culturing a population of pluripotent stem cells, the method comprising:
culturing in a first medium comprising one or more inhibitors of GSK-3β, one or more inhibitors of JNK, one or more inhibitors of p38, one or more inhibitors of PKC and one or more inhibitors of Erk1/2; and
culturing in a second medium comprising one or more inhibitors of GSK-3β, one or more inhibitors of JNK, one or more inhibitors of p38 and one or more inhibitors of PKC, and not comprising an inhibitor of Erk1/2.

2. The method of claim 1, wherein the first and second media further comprise one or more of fibroblast growth factor 2; transforming growth factor-β1; a STAT3 activator; insulin; ascorbic acid; albumin; N2 supplement; non-essential amino acids; glutamine; and serum replacement.

3. The method of claim 1, wherein the pluripotent stem cells cultured in the second medium are cultured as aggregates.

4. The method of claim 1, wherein the second medium further comprises a polysulfated compound

5. The method of claim 1, wherein the growth rate of the pluripotent stem cells cultured in the first medium and the second medium is increased compared to pluripotent stem cells not cultured in the first medium and the second medium.

6. A method for culturing a population of pluripotent stem cells, the method comprising culturing the pluripotent stem cells in a medium comprising a polysulfated compound.

7. The method of claim 6, wherein the pluripotent stem cells are cultured as aggregates.

8. The method of claim 6, wherein the polysulfated compound is dextran sulfate.

9. The method of claim 6, wherein the dextran sulfate has a molecular weight of about 4,000 Da to about 500,000 Da.

10. The method of claim 6, the method further comprising culturing the pluripotent stem cell aggregates under dynamic suspension conditions.

11. The method of claim 6, wherein the pluripotent stem cells are human.

12. The method of claim 6, wherein the pluripotent stem cells are differentiated to cardiac progenitor cells, cardiac cells, pancreatic progenitor cells, pancreatic cells, ectoderm cells, neural cells, or hematopoietic cells.

13. A medium for culturing pluripotent stem cell, the medium comprising a polysulfated compound.

14. The medium of claim 13, wherein the pluripotent stem cells are cultured as aggregates.

15. The medium of claim 13, wherein the polysulfated compound is dextran sulfate.

16. The medium of claim 13, wherein the dextran sulfate has a molecular weight of about 4,000 Da to about 500,000 Da.

17. The medium of claim 13, wherein the pluripotent stem cells are human.

18. The method of claim 6 wherein the method generates at least one isolated single pluripotent stem cell.

19. The medium of claim 13 wherein the medium generates at least one isolated single pluripotent stem cell.

20. The method of claim 18 wherein the at least one isolated single pluripotent stem cell is human.
